(19) United States
(12) Patent Application Publication
(10) Pub. No.: US 2024/0350447 A1
(43) Pub. Date: Oct. 24, 2024

(54) DELIVERY METHODS FOR TREATING BRAIN AND CENTRAL NERVOUS SYSTEM DISEASES

(71) Applicant: JENIVISION INC., Irvine, CA (US)

(72) Inventors: Weizhen WANG, Irvine, CA (US); Nonna SNIDER, Irvine, CA (US)

(21) Appl. No.: 18/687,258

(22) PCT Filed: Nov. 23, 2022
(86) PCT No.: PCT/US2022/080453

§ 371 Date: Feb. 27, 2024

Related U.S. Application Data
(60) Provisional application No. 63/401,918, filed on Aug. 29, 2022.
(60) Provisional application No. 63/283,121, filed on Nov. 24, 2021.

(51) Int. Cl. A61K 31/202; A61K 9/00; A61K 31/232; A61K 31/355; A61K 35/60; A61K 47/44

(52) U.S. Cl. CPC A61K 31/202; A61K 9/0014; A61K 9/0048; A61K 31/232; A61K 31/355; A61K 35/60; A61K 47/44

(57) ABSTRACT

Described herein are compositions and methods for the topical administration of various compounds to the periorbital and eyelid region of the eye. Such compositions and methods are useful for treating various brain and central nervous system diseases and disorders, as well as in promoting general brain and central nervous system health.

### CROSS REFERENCE

This application claims the benefit of U.S. Provisional Application No. 63/283,121 filed Nov. 24, 2021 and U.S. Provisional Application No. 63/401,918 filed Aug. 29, 2022, both of which are incorporated herein by reference in entirety.

### TECHNICAL FIELD

Embodiments herein are directed towards the delivery of compounds to the brain, central nervous system (CNS) and other parts of the body via a non-invasive ocular delivery platform (NIODP) applied through the periorbital skin or eyelid skin of a patient to improve health and treat diseases.

### SUMMARY

Provided herein are methods and compositions for delivery of compounds, including omega-3 fatty acids, via periorbital or eyelid administration to the brain/CNS. Surprisingly, it has been found herein that omega-3 fatty acids, including docosahexaenoic acid (DHA), can be effectively delivered to the brain/CNS via periorbital or eyelid administration.

In an aspect, provided herein is a method of promoting health, preventing or treating disease in a brain of a subject, comprising administering a topical pharmaceutical composition comprising an omega-3 fatty acid to the periorbital skin or eyelid of an eye of the subject.

In some cases, the omega-3 fatty acid is isolated from fish tissue. In some cases, the omega-3 fatty acid is isolated from a plant source. In some cases, the omega-3 fatty acid comprises alpha-linolenic acid (ALA), eicosapentaenoic acid (EPA), docosahexaenoic acid (DHA), or any combination thereof. In some cases, the omega-3 fatty acid comprises DHA.

In some cases, the topical pharmaceutical composition comprises greater than 50% DHA. In some cases, the topical pharmaceutical composition comprises greater than 75% DHA. In some cases, the topical pharmaceutical composition comprises greater than 90% DHA. In some cases, the topical pharmaceutical composition comprises greater than 95% DHA. In some cases, the topical pharmaceutical composition comprises greater than 99% DHA.

In some cases, the topical pharmaceutical composition comprises petrolatum. In some cases, the topical pharmaceutical composition comprises shea butter. In some cases, the topical pharmaceutical composition comprises lanolin. In some cases, the topical pharmaceutical composition comprises Vitamin E. In some cases, the topical pharmaceutical composition comprises less than 0.2% Vitamin E.

In some cases, the omega-3 fatty acid is administered in an amount of from about 0.1 mg to about 3000 mg, about 0.1 mg to about 1000 mg, about 0.1 mg to about 500 mg, about 0.1 mg to about 200 mg, or about 0.1 mg to about 100 mg. In some cases, the omega-3 fatty acid is administered in an amount of 5 to 10 mg per eye.

In some cases, the topical pharmaceutical composition is formulated as a cream, emulsion, ointment, or oil solution. In some cases, the topical pharmaceutical composition further comprises an emollient, a humectant, a thickening agent, a preservative, a penetration enhancer, an anti-oxidant, an odor masking agent, or any combination thereof. In some cases, the topical pharmaceutical composition further comprises a preservative. In some cases, the topical pharmaceutical composition is free of preservatives.

In some cases, the topical pharmaceutical composition is administered with a bottle with a roller ball, a click pen brush, a pump bottle, or an eye drop bottle and Q-tip, an eye pad, or a fingertip.

In some cases, promoting health, preventing or treating disease in a brain of a subject comprises treating or preventing addiction, arachnoid cysts, attention deficit-hyperactivity disorder, autism, brain injury, brain tumor, catalepsy, encephalitis, epilepsy, meningitis, migraine, multiple sclerosis, myelopathy, Tourette's syndrome, Alzheimer's disease, Huntington's disease, or Parkinson's disease.

In some cases, the topical pharmaceutical composition is administered to the subject four times per day, three times per day, twice per day, once per day, once every other day, once every three days, once every four days, or once every seven days.

In another aspect, provided herein is a method for assessing the safety profile of an article, comprising: administering a topical composition comprising the article to the periorbital skin or eyelid of an eye of a subject and measuring a concentration of the article in a tissue of the undosed eye, brain, or central nervous system of the subject, wherein if the concentration of the article is above a threshold value, the dosed article is determined to be acceptable for administration to the brain or central nervous system. In some cases, the tissue of the undosed eye comprises retina tissue.

In another aspect, provided herein is a method of delivering an omega-3 fatty acid to a brain of a subject, comprising administering a topical pharmaceutical composition comprising the omega-3 fatty acid to periorbital skin or eyelid of an eye of the subject, wherein greater than 0.05% of the omega-3 fatty acid administered to the periorbital skin or eyelid of the subject is delivered to the brain of the subject.

In some cases, greater than 0.1% of the omega-3 fatty acid administered to the periorbital skin or eyelid of the subject is delivered to the brain of the subject. In some cases, greater than 0.5% of the omega-3 fatty acid administered to the periorbital skin or eyelid of the subject is delivered to the brain of the subject. In some cases, greater than 1.0% of the omega-3 fatty acid administered to the periorbital skin or eyelid of the subject is delivered to the brain of the subject. In some cases, greater than 4.0% of the omega-3 fatty acid administered to the periorbital skin or eyelid of the subject is delivered to the brain of the subject.

In some cases, the omega-3 fatty acid is isolated from fish tissue. In some cases, the omega-3 fatty acid is isolated from a plant source. In some cases, the omega-3 fatty acid comprises alpha-linolenic acid (ALA), eicosapentaenoic acid (EPA), docosahexaenoic acid (DHA), or any combination thereof. In some cases, the omega-3 fatty acid comprises DHA. In some cases, the topical pharmaceutical composition comprises greater than 50% DHA. In some cases, the topical pharmaceutical composition comprises greater than 75% DHA. In some cases, the topical pharmaceutical composition comprises greater than 90% DHA. In some cases, the topical pharmaceutical composition comprises greater than 95% DHA. In some cases, the topical pharmaceutical composition comprises greater than 99% DHA. In some cases, the topical pharmaceutical composition comprises petrolatum. In some cases, the topical pharmaceutical composition comprises shea butter. In some cases, the topical pharmaceutical composition comprises lanolin. In some cases, the topical pharmaceutical composition comprises Vitamin E. In some cases, wherein the topical pharmaceutical composition comprises less than 0.2% Vitamin E.

In some cases, the omega-3 fatty acid is administered in an amount of from about 0.1 mg to about 3000 mg, about 0.1 mg to about 1000 mg, about 0.1 mg to about 500 mg, about 0.1 mg to about 200 mg, about 0.1 mg to about 100 mg, or about 0.1 mg to about 30 mg. In some cases, the omega-3 fatty acid is administered in an amount of 5 to 10 mg per eye.

In some cases, the topical pharmaceutical composition is formulated as a cream, emulsion, ointment, or oil solution. In some cases, the topical pharmaceutical composition further comprises an emollient, a humectant, a thickening agent, a preservative, a penetration enhancer, an anti-oxidant, an odor masking agent, or any combination thereof. In some cases, the topical pharmaceutical composition further comprises a preservative. In some cases, the topical pharmaceutical composition is free of preservatives.

In some cases, the topical pharmaceutical composition is administered with a bottle with a roller ball, a click pen brush, a pump bottle, or an eye drop bottle and Q-tip, an eye pad, or a fingertip.

In some cases, delivery of the topical pharmaceutical composition to the brain of the subject prevents or treats a brain disease of the subject. In some cases, the brain diseases comprises addiction, arachnoid cysts, attention deficit-hyperactivity disorder, autism, brain injury, brain tumor, catalepsy, encephalitis, epilepsy, meningitis, migraine, multiple sclerosis, myelopathy, Tourette's syndrome, Alzheimer's disease, Huntington's disease, or Parkinson's disease.

In some cases, the topical pharmaceutical composition is administered to the subject four times per day, three times per day, twice per day, once per day, once every other day, once every three days, once every four days, or once every seven days.

In some cases, the pharmaceutical composition comprises one or more emollients in an amount of less than 95% (w/w) of the composition. In some cases, the pharmaceutical composition comprises one or more emollients in an amount of less than 50% (w/w) of the composition. In some cases, the pharmaceutical composition comprises the omega-3 fatty acid in an amount of at least 5% (w/w) of the composition. In some cases, the pharmaceutical composition comprises the omega-3 fatty acid in an amount of at least 50% (w/w) of the composition. In some cases, the pharmaceutical composition comprises the omega-3 fatty acid in an amount of at least 95% (w/w) of the composition.

In some cases, the pharmaceutical composition further comprises Vitamin E. In some cases, the pharmaceutical composition comprises the Vitamin E in an amount of less than 0.3% (w/w) of the composition.

In another aspect, provided herein is a pharmaceutical composition formulated for topical periorbital or eyelid administration, comprising an omega-3 fatty acid and one or more emollients.

In some cases, the omega-3 fatty acid is isolated from fish tissue. In some cases, the omega-3 fatty acid is isolated from a plant source. In some cases, the omega-3 fatty acid comprises alpha-linolenic acid (ALA), eicosapentaenoic acid (EPA), docosahexaenoic acid (DHA), or any combination thereof. In some cases, the omega-3 fatty acid comprises docosahexaenoic acid (DHA). In some cases, the pharmaceutical composition comprises greater than 50% DHA. In some cases, the pharmaceutical composition comprises greater than 75% DHA. In some cases, the pharmaceutical composition comprises greater than 90% DHA. In some cases, the pharmaceutical composition comprises greater than 95% DHA. In some cases, the pharmaceutical composition comprises greater than 99% DHA.

In some cases, the one or more emollients comprises petrolatum. In some cases, the one or more emollients comprises shea butter. In some cases, the one or more emollients comprises lanolin. In some cases, the pharmaceutical composition comprises Vitamin E. In some cases, the pharmaceutical composition comprises less than 0.2% Vitamin E.

In some cases, the pharmaceutical composition is formulated as a cream, emulsion, ointment, or oil solution. In some cases, the pharmaceutical composition further comprises a humectant, a thickening agent, a preservative, a penetration enhancer, an anti-oxidant, an odor masking agent, or any combination thereof. In some cases, the pharmaceutical composition further comprises a preservative. In some cases, the pharmaceutical composition is free of preservatives.

In some cases, the pharmaceutical composition is administered with a bottle with a roller ball, a click pen brush, a pump bottle, or an eye drop bottle and Q-tip, an eye pad, or a fingertip.

In some cases, the topical pharmaceutical composition is administered to periorbital skin or an eyelid of a subject four times per day, three times per day, twice per day, once per day, once every other day, once every three days, once every four days, or once every seven days.

In some cases, the pharmaceutical composition comprises one or more emollients in an amount of less than 95% (w/w) of the composition. In some cases, the pharmaceutical composition comprises one or more emollients in an amount of less than 50% (w/w) of the composition. In some cases, the pharmaceutical composition comprises one or more emollients in an amount of less than 5% (w/w) of the composition. In some cases, the pharmaceutical composition comprises the omega-3 fatty acid in an amount of at least 5% (w/w) of the composition. In some cases, the pharmaceutical composition comprises the omega-3 fatty acid in an amount of at least 50% (w/w) of the composition. In some cases, the pharmaceutical composition comprises the omega-3 fatty acid in an amount of at least 95% (w/w) of the composition.

In some cases, the pharmaceutical composition further comprises Vitamin E. In some cases, the pharmaceutical composition comprises the Vitamin E in an amount of less than 0.3% (w/w) of the composition.

In some cases, the pharmaceutical composition further comprises greater than 3000 μg of the omega-3 fatty acid. In some cases, the pharmaceutical composition further comprises greater than 5000 μg of the omega-3 fatty acid. In some cases, the pharmaceutical composition further comprises greater than 6000 μg of the omega-3 fatty acid.

In another aspect, provided herein is a method of delivering an omega-3 fatty acid to a brain of a subject, comprising administering a topical pharmaceutical composition comprising the omega-3 fatty acid to periorbital skin or eyelid of the subject, wherein after administration, a concentration of the omega-3 fatty acid in the brain of the subject is greater than 100 nmol/g of tissue.

In some cases, after administration, the concentration of the omega-3 fatty acid in the brain of the subject is greater than 110 nmol/g of tissue.

In some cases, the omega-3 fatty acid comprises DHA. In some cases, the topical pharmaceutical composition comprises greater than 50% DHA. In some cases, the topical pharmaceutical composition comprises greater than 75% DHA. In some cases, the topical pharmaceutical composition comprises greater than 90% DHA. In some cases, the topical pharmaceutical composition comprises greater than 95% DHA. In some cases, the topical pharmaceutical composition comprises greater than 99% DHA.

In some cases, the topical pharmaceutical composition comprises petrolatum. In some cases, the topical pharmaceutical composition comprises shea butter. In some cases, the topical pharmaceutical composition comprises lanolin. In some cases, the topical pharmaceutical composition comprises Vitamin E. In some cases, the topical pharmaceutical composition comprises less than 0.2% Vitamin E.

In some cases, the omega-3 fatty acid is administered in an amount of from about 0.1 mg to about 3000 mg, about 0.1 mg to about 1000 mg, about 0.1 mg to about 500 mg, about 0.1 mg to about 200 mg, about 0.1 mg to about 100 mg, or about 0.1 mg to about 30 mg. In some cases, the omega-3 fatty acid is administered in an amount of 5 to 10 mg per eye.

In some cases, the topical pharmaceutical composition is formulated as a cream, emulsion, ointment, or oil solution. In some cases, the topical pharmaceutical composition further comprises an emollient, a humectant, a thickening agent, a preservative, a penetration enhancer, an anti-oxidant, an odor masking agent, or any combination thereof. In some cases, the topical pharmaceutical composition further comprises a preservative. In some cases, the topical pharmaceutical composition is free of preservatives. In some cases, the topical pharmaceutical composition is administered with a bottle with a roller ball, a click pen brush, a pump bottle, or an eye drop bottle and Q-tip, an eye pad, or a fingertip.

In some cases, delivery of the topical pharmaceutical composition to the brain of the subject prevents or treats a brain disease of the subject. In some cases, the brain diseases comprises addiction, arachnoid cysts, attention deficit-hyperactivity disorder, autism, brain injury, brain tumor, catalepsy, encephalitis, epilepsy, meningitis, migraine, multiple sclerosis, myelopathy, Tourette's syndrome, Alzheimer's disease, Huntington's disease, or Parkinson's disease.

In some cases, the topical pharmaceutical composition is administered to the subject four times per day, three times per day, twice per day, once per day, once every other day, once every three days, once every four days, or once every seven days.

In some cases, the pharmaceutical composition comprises one or more emollients in an amount of less than 95% (w/w) of the composition. In some cases, the pharmaceutical composition comprises one or more emollients in an amount of less than 50% (w/w) of the composition. In some cases, the pharmaceutical composition comprises one or more emollients in an amount of less than 5% (w/w) of the composition. In some cases, the pharmaceutical composition comprises the omega-3 fatty acid in an amount of at least 5% (w/w) of the composition. In some cases, the pharmaceutical composition comprises the omega-3 fatty acid in an amount of at least 50% (w/w) of the composition. In some cases, the pharmaceutical composition comprises the omega-3 fatty acid in an amount of at least 95% (w/w) of the composition.

In some cases, the pharmaceutical composition further comprises Vitamin E. In some cases, the pharmaceutical composition comprises the Vitamin E in an amount of less than 0.3% (w/w) of the composition.

### INCORPORATION BY REFERENCE

All publications, patents, and patent applications mentioned in this specification are herein incorporated by reference to the same extent as if each individual publication, patent, or patent application was specifically and individually indicated to be incorporated by reference.

### BRIEF DESCRIPTION OF THE FIGURES

FIG. 1 illustrates a schematic diagram of the optic nerve in the brain.

FIG. 2 illustrates the model of forward and reverse trans-ocular/retinal transport (TORT) pathway in the CNS.

FIG. 3 shows the anatomy of the periorbital region of the eye.

### DETAILED DESCRIPTION

Provided herein are methods and compositions for delivery of compounds, including omega-3 fatty acids, via periorbital or eyelid administration. Surprisingly, it has been found herein that omega-3 fatty acids, including docosahexaenoic acid (DHA), can be effectively delivered to and the brain/CNS and systemically throughout the body via periorbital or eyelid administration.

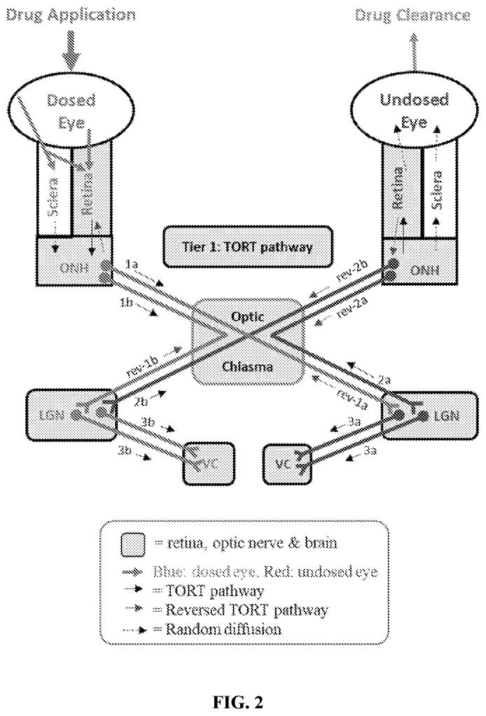

Drug delivery through the blood-brain barrier (BBB) remains a major challenge. Great efforts and various strategies have been exploited to deliver drugs and diagnostic agents into the brain. The various limitations, including low efficiency of crossing BBB, invasiveness of brain injection or intravenous (IV) administration, failure to translate from rodents to humans, and brain toxicity and long-term safety concerns are difficult to overcome. Recently under investigation is the nose-to-brain route, where mechanisms are still not clearly understood, with travel across nerve fibers and travel via a perivascular pathway both being hypothesized. Based on the discovery in the systems and methods described herein, the mechanism of nose-to-brain route would include traveling across nerve fibers via the olfactory nerve system to the brain, then through the cerebrospinal fluid (CSF), and lastly through blood circulation. Unlike the rev-TORT pathway proposed in FIG. 2 for the optic nerve system, there may not be direct material recycling in the olfactory system since the left and right olfactory tract is not crossed. Additionally, since the nose-to-brain route has been demonstrated in human studies with solution and particle formulations, the NIODP route may also be able to deliver solutions and well formulated particles to the brain. The NIODP delivery seems to be much easier to execute, since the nose-to-brain dose must be deposited in the olfactory nerve region but not to the respiratory region, and thus, a special delivery device is required.

Briefly, CNS drug delivery, efficacy and safety are great challenges, which cause significantly longer development times, longer post-development regulatory review, and often complete failure, despite great efforts. The CNS drug attrition rate is extremely high. In major neurodegenerative diseases, such as Alzheimer's disease (AD) and Parkinson's disease (PD) that affect millions, the clinical failure rate has been 100% for disease-modifying treatments. There is, therefore, an urgent need for new approaches in CNS Drug development and delivery.

A Non-Invasive Ocular Delivery Platform (NIODP) can open a brand-new pathway to treat brain and systemic diseases. Delivery via the periorbital or eyelid skin may present a brain/CNS drug delivery pathway to treat causes in addition to treat symptoms of brain/CNS diseases. Drugs or any active ingredients that reach the retina, via NIODP or other routes, may go through the optic nerve via the Trans-ocular/retinal transport (TORT) pathway for optic nerve recycle or clearance (FIG. 2). NIODP may deliver drugs and active ingredients to the brain/CNS via the TORT.

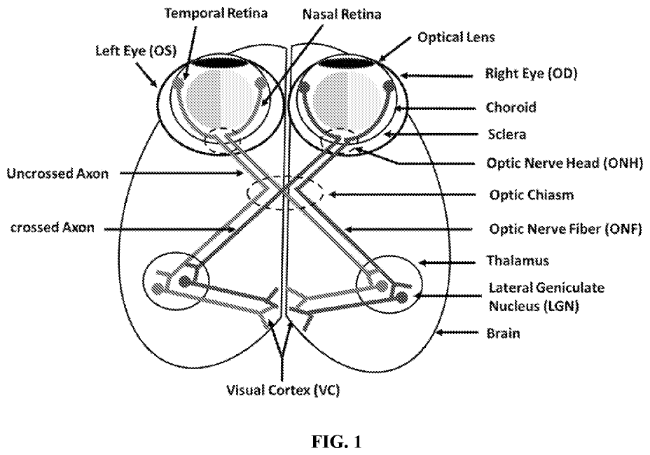

FIG. 1 depicts a schematic diagram of the optic nerve in the brain. The optic nerve connects the retina to the brain. The optic nerve begins from the optic nerve head (ONH), which is the merging point of the posterior retina, choroid and sclera, and the starting point of the cerebrospinal fluid (CSF) space of the ocular globe. The optic nerve also includes the Optic chiasm, an X-shaped structure formed by the crossed and uncrossed axon or optic nerve fibers (OBF). The optic nerve also includes the Lateral Geniculate Nucleus (LGN), where most of the neuron synapse, which is a relay center in the thalamus, receiving and transporting input from both eyes via the crossed and uncrossed axon to the cerebral visual cortex (VC).

After administration via NIODP or other route to the retina, certain materials may be readily accepted to stay travelalong the optic nerve for CNS (retina and brain) distribution and absorption via the TORT pathway. For molecules and particles to stay in the CNS, they need to meet certain requirements, such as having proper binding proteins, etc. to pass through a complex sorting system. If those requirements are not met, the materials may be identified as “waste” to be quickly removed by ATP-binding cassette efflux transporters are strongly expressed in brain capillaries. The efflux transporters limit CNS uptake of certain molecules and protect the CNS from exposure to circulating chemicals. The forward TORT pathway (solid black arrows, FIG. 2) provides a fast one-way-out route for any “waste” molecules managed to penetrate the CNS to be quickly expelled out of the CNS to various outlets nearby, such as the non-CNS tissues along the forward TORT pathway, including the ocular tissues/muscles of the undosed eye, the CSF, and/or blood for terminal clearance. On the other hand, as depicted in FIG. 2, after administration via NIODP or other route to the retina, “acceptable” molecules and particles may travel and circulate along the optic nerve in the CNS as the following: From the undosed or higher dose side of retina to ONH, through axonal transport via the nasal retinal optic nerve fiber (crossed axon, 1a) to the contralateral side of the LGN, then through synaptic transmission via the uncrossed axon (2a), to the contralateral ONH and retina.
and at the same time, through axonal transport via the temporal retinal optic nerve fiber (uncrossed axon, 1b) to the LGN of the same side, through synaptic transmission via the crossed axon (2b) to the ONH and retina of the contralateral side.
and from both LGN, following the black arrows (3a and 3b) through synaptic transmission in both LGN to both visual cortexes (VC).

The proposed reversed TORT pathway (rev-TORT), described as in FIG. 2, may keep a constant homeostasis system for quick balance and constant equilibrium of concentration of molecular/particle that identified as “acceptable”, such as essential nutrients between the retinas and in the optic nerve system. The rev-TORT may include from the retina and ONH of the contralateral eye by following the red arrows rev-2a or rev-3a through rev-1a, and rev-2b or rev-3b through rev-1b to the original ONH and retina. The forward and reverse TORT pathway may be used for delivery of brain therapeutics, particularly for essential nutrients.

After saturating the retina tissue and the rest of optic nerve system, surplus of the “acceptable” material, if any, which cannot be completely absorbed by the CNS, may be spilled out to the surrounding tissues/muscles along the TORT pathway for use or clearance.

A drug or formulation being readily accepted to remain and recycle in the TORT pathway at high doses may indicate that the drug or formulation is safe for the brain and/or central nervous system. Therefore, the TORT pathway can be used to screen for drugs or formulations that are safe for the brain or central nervous system, regardless of delivery method (oral, intravenous injection, nose-to-brain, and/or brain injection, for example). In some cases, a drug or formulation can be delivered via NIODP (either to the eyelid or periorbital skin) to assess its long-term safety profile. In some cases, a drug of formulation can be delivered by ocular injection (including subconjunctival, suprachoroidal, subretinal, and trans-scleral injections) to assess its long term-safety profile. Any delivery method that results in the drug or formulation being delivered to the retina of one eye of the subject may be used. In some cases, a drug or formulation can be delivered to the eyelid or periorbital skin to assess its long-term safety profile in the brain and/or CNS. A drug or formulation that is readily accepted to remain and recycle in the TORT pathway may be safe for the brain and/or CNS. A drug or formulation that is not readily accepted to remain and recycle in the TORT pathway and is instead quickly cleared out from the dosed retina may be unsafe for the brain and/or CNS. In some cases, a method for assessing the safety profile of an article comprises administering a topical composition comprising the article to the periorbital skin or eyelid of an eye of a subject and measuring a concentration of the article in a tissue of the undosed retina, brain, or central nervous system of the subject. In some cases, a method for assessing the safety profile of an article comprises an ocular injection (including subconjunctival, suprachoroidal, subretinal, and trans-scleral injections) of a formulation comprising the article to an eye of the subject and measuring a concentration of the article in a tissue of the undosed retina, brain, or central nervous system of the subject. If the concentration of the article is above a threshold value, the dosed article may be determined acceptable for administration to the brain or central nervous system. If a quick balance and constant equilibrium of concentration of the article is demonstrated between the dosed and undosed eyes of the retina, the dosed article may be determined acceptable for administration to the brain or central nervous system.

### Diseases of Brain and Central Nervous System

Compared to the current invasive brain injection or intravenous (IV) administration for brain drugs, non-invasive NIODP delivery may reduce treatment burden on patients and may require a lower dose of an active ingredient to deliver therapeutically adequate amounts both to the central nervous system and systemically. Embodiments disclosed are directed towards the treatment of diseases of the central nervous system.

In some embodiments, the central nervous system disease or disorder comprises brain inflammation. In some embodiments, the brain inflammation is caused by a foreign substance or viral infection. In some embodiments, the central nervous system disease or disorder comprises an infection. In some embodiments, the infection is caused by a virus, bacteria, protozoa, fungi, prion, or a combination thereof. In some embodiments, the disease or disorder comprises a neurodegenerative disease or disorder. In some embodiments, the neurodegenerative disease or disorder comprises Alzheimer's disease, Huntington's disease, or Parkinson's disease, or a combination thereof. In some embodiments, the disease or disorder comprises addiction, arachnoid cysts, attention deficit-hyperactivity disorder, autism, catalepsy, encephalitis, epilepsy, locked-in syndrome, meningitis, migraine, multiple sclerosis, myelopathy, or Tourette's syndrome, or a combination thereof. In some embodiments, the disease or disorder is caused by a brain injury or brain tumor.

Formulations applied to the NIODP to treat diseases of the brain and CNS may comprise chlorpromazine, haloperidol, amisulpiride, levomepromazine, norclozapine, olanzapine, paliperidone, quetiapine, risperidone, sulpiride, triapride, ziprasidone, thioridazine, aripiprazole, clozapine, flupentixol, melperone, perphenazine, pimozide, raclopride, thioxanthene, trifluperazine hydrochloride, or ziprasidone, C-Donepezil, Donepezil, Memantine, Rivastigmine, or Galantamine, Efavirenz, Atazanavir, Emtricitabine, Lamivudine, Lopinavir, or Tenofovir, or a combination thereof.

Addiction is a disorder of the brain's reward system that arises through transcriptional and epigenetic mechanisms and occurs over time from chronically high levels of exposure to an addictive stimulus (e.g., morphine, cocaine, sexual intercourse, gambling, etc.). In some embodiments, formulations can be delivered through NIODP that correct malfunctioning transcriptional and epigenetic mechanisms and therefore treat or minimize the severity of addiction.

In some embodiments, formulations may be delivered through NIODP to treat arachnoid cysts. Arachnoid cysts are cerebrospinal fluid covered by arachnoidal cells that may develop on the brain or spinal cord. They are a congenital disorder, and in some cases may not show symptoms. However, if there is a large cyst, symptoms may include headache, seizures, ataxia (lack of muscle control), hemiparesis, and several others. Macrocephaly and ADHD are common among children, while presenile dementia, hydrocephalus (an abnormality of the dynamics of the cerebrospinal fluid), and urinary incontinence are symptoms for elderly patients (65 and older).

In some embodiments, formulations may be delivered through NIODP to treat attention deficit-hyperactivity disorder (ADHD). ADHD is an organic disorder of the nervous system. ADHD, which in severe cases can be debilitating, has symptoms thought to be caused by structural as well as biochemical imbalances in the brain; in particular, low levels of the neurotransmitters dopamine and norepinephrine, which are responsible for controlling and maintaining attention and movement. Many people with ADHD continue to have symptoms well into adulthood. Also of note is an increased risk of the development of Dementia with Lewy bodies, or (DLB), and a direct genetic association of ADHD to Parkinson's disease two progressive, and serious, neurological diseases whose symptoms often occur in people over age 65.

In some embodiments, formulations may be delivered through NIODP to treat autism. Autism is a neurodevelopmental disorder that is characterized by restricted and repetitive patterns of behavior and persistent deficits in social interaction and communication.

In some embodiments, formulations may be delivered through NIODP to treat brain injuries or brain tumors. Tumors of the central nervous system constitute around 2% of all cancer in the United States.

In some embodiments, formulations may be delivered through NIODP to treat Catalepsy. Catalepsy is a nervous disorder characterized by immobility and muscular rigidity, along with a decreased sensitivity to pain. Catalepsy is considered a symptom of serious diseases of the nervous system (e.g., Parkinson's disease, Epilepsy, etc.) rather than a disease by itself. Cataleptic fits can range in duration from several minutes to weeks. Catalepsy often responds to Benzodiazepines (e.g., Lorazepam) in pill and I.V. form.

In some embodiments, formulations may be delivered through NIODP to treat Encephalitis. Encephalitis is an inflammation of the brain. It is usually caused by a foreign substance or a viral infection. Symptoms of this disease include headache, neck pain, drowsiness, nausea, and fever. If caused by the West Nile virus, and it may be lethal to humans, as well as birds and horses.

In some embodiments, formulations may be delivered through NIODP to treat Epilepsy. Epilepsy is an unpredictable, serious, and potentially fatal disorder of the nervous system, thought to be the result of faulty electrical activity in the brain. Epileptic seizures result from abnormal, excessive, or hypersynchronous neuronal activity in the brain. About 50 million people worldwide have epilepsy, and nearly 80% of epilepsy occurs in developing countries. Epilepsy becomes more common as people age. Onset of new cases occurs most frequently in infants and the elderly. Epileptic seizures may occur in recovering patients as a consequence of brain surgery.

In some embodiments, formulations may be delivered through NIODP to treat infections of the CNS. A number of different pathogens (i.e., certain viruses, bacteria, protozoa, fungi, and prions) can cause infections that adversely affect the brain or spinal cord.

In some embodiments, formulations may be delivered through NIODP to treat locked-in syndrome. Locked-in syndrome usually results from a stroke that damages part of the brainstem, in which the body and most of the facial muscles are paralyzed but consciousness remains and the ability to perform certain eye movements is preserved.

In some embodiments, formulations may be delivered through NIODP to treat meningitis. Meningitis is an inflammation of the meninges (membranes) of the brain and spinal cord. It is most often caused by a bacterial or viral infection. Fever, vomiting, and a stiff neck are all symptoms of meningitis.

In some embodiments, formulations may be delivered through NIODP to treat migraines. A migraine is a chronic, often debilitating neurological disorder characterized by recurrent moderate to severe headaches, often in association with a number of autonomic nervous system symptoms.

In some embodiments, formulations may be delivered through NIODP to treat multiple sclerosis (MS). MS is a chronic, inflammatory demyelinating disease, meaning that the myelin sheath of neurons is damaged. Symptoms of MS include visual and sensation problems, muscle weakness, numbness and tingling all over, muscle spasms, poor coordination, and depression. Also, patients with MS have reported extreme fatigue and dizziness, tremors, and bladder leakage.

In some embodiments, formulations may be delivered through NIODP to treat myelopathy. Myelopathy is an injury to the spinal cord due to severe compression that may result from trauma, congenital stenosis, degenerative disease or disc herniation. The spinal cord is a group of nerves housed inside the spine that runs almost its entire length.

In some embodiments, formulations may be delivered through NIODP to treat Tourette's syndrome. Tourette's syndrome is an inherited neurological disorder. Early onset may be during childhood, and it is characterized by physical and verbal tics. Tourette's often also includes symptoms of both OCD and ADHD indicating a link between the three disorders. The exact cause of Tourette's, other than genetic factors, is unknown.

In some embodiments, formulations may be delivered through NIODP to treat or relieve symptoms associated with brain cancer. DHA has anti-inflammation, anti-neovascularization, and analgesic properties which can be beneficial to treat or reduce symptoms associated with brain cancer. In some cases, DHA can be delivered via NIODP to treat or relieve symptoms associated with brain cancer. DHA can be administered via NIODP as a palliative/supportive care for patients undergoing cancer treatment of either primary brain tumor or with brain tumor metastasis. DHA, administered via NIODP, may provide nutrition support directly to the brain tissue, therefore facilitating recovery of the healthy tissue and combatting toxicities of cancer treatments. Administration of DHA via NIODP to cancer patients therefore might can improve quality of life and shorten the recovery time. For patients undergoing chemotherapy, DHA may be beneficial when administered during the treatment and maintenance phases.

In some cases, DHA can be crosslinked with a small molecule drug to form a DHA-crosslinked small molecule drug (DCSMD) and delivered via NIODP to treat or relieve symptoms associated with brain cancer. A DCSMD may help drug penetration via periorbital delivery to the brain. DCSMD may increase drug retention in the brain so that frequency of dosing can be reduced. DHA or the drug of the DCSMD may or may not need to be released by enzymatic cleavage to be an active drug.

### Neuroprotection and Neurodegenerative Disorders

Formulations capable of delivering therapeutically adequate amounts of the compound to the central nervous system and blood are included, achieved by application to the periorbital or eyelid skin. Embodiments disclosed are directed towards the treatment of neurodegenerative disorders.

In some embodiments, DHA or any other omega-3 compound may be applied via NIODP for neuroprotection and to treat neurodegenerative diseases. Because of its well tolerated safety profile and mechanism of action for anti-inflammation and neuroprotection, DHA may be a potential therapeutic agent for age-related neurodegenerative diseases. Studies in animal models suggest that above normal nutritional requirement intake of DHA might modify the risk/course of some diseases of the brain. Epidemiological studies suggest that DHA/omega-3 fatty acid dietary supplement may reduce the risk of Alzheimer disease (AD) and Parkinson's diseases (PD).

In some embodiments, formulations may be delivered through NIODP to treat Alzheimer's disease. Alzheimer's is a neurodegenerative disease typically found in people over the age of 65 years. Worldwide, approximately 24 million people have dementia; 60% of these cases are due to Alzheimer's. The ultimate cause is unknown. The clinical sign of Alzheimer's is progressive cognition deterioration. Formulations comprising C-Donepezil, Donepezil, Memantine, Rivastigmine, or Galantamine can be delivered through NIODP to treat Alzheimer's disease.

In some embodiments, formulations may be delivered through NIODP to treat Huntington's disease. Huntington's disease is a degenerative neurological disorder that is inherited. Degeneration of neuronal cells occurs throughout the brain, especially in the striatum. There is a progressive decline that results in abnormal movements. Statistics show that Huntington's disease may affect 10 per 100,000 people of Western European descent.

In some embodiments, formulations may be delivered through NIODP to treat Parkinson's disease. Parkinson's disease, or PD, is a progressive illness of the nervous system. Caused by the death of dopamine-producing brain cells that affect motor skills and speech. Symptoms may include bradykinesia (slow physical movement), muscle rigidity, and tremors. Behavior, thinking, sensation disorders, and the sometimes co-morbid skin condition Seborrheic dermatitis are just some of PD's numerous nonmotor symptoms. Parkinson's disease, Attention deficit/hyperactivity disorder (ADHD) and Bi-polar disorder, all appear to have some connection to one another, as all three nervous system disorders involve lower than normal levels of the brain chemical dopamine (In ADHD, Parkinson's, and the depressive phase of Bi-polar disorder.) or too much dopamine (in Mania or Manic states of Bi-polar disorder) in different areas of the brain.

### Hair Growth Disorders

Formulations applied to the periorbital or eyelid skin (NIODP) may be capable of delivering therapeutically adequate amounts of a compound to systemic circulation to treat hair growth disorders. Embodiments disclosed are directed towards hair growth disorders. DHA or any other omega-3 compound may be applied via NIODP to treat hair growth disorders. Hair growth disorders may comprise androgenetic alopecia (male or female pattern baldness), alopecia areata, scarring (cicatricial) alopecia, telogen effluvium, anagen effluvium, congenital hypotrichosis, or infection-related hair loss such as tinea capitis and folliculitis. Omega-3 supplementation via delivery to the periorbital skin or to the eyelid may improve hair density and reduce the telogen percentage and the proportion of miniaturized anagen hair.

### Autoimmune Disorders

Formulations applied to the periorbital or eyelid skin (NIODP) may be capable of delivering therapeutically adequate amounts of a compound to systemic circulation to treat autoimmune disorders including Addison disease, celiac disease, dermatomyositis, Graves disease, Hashimoto thyroiditis, multiple sclerosis, myasthenia gravis, pernicious anemia, Sjogren syndrome, systemic lupus erythematosus, Type I diabetes, Guillain-Barre syndrome, and scleroderma. Formulations delivered via NIODP to treat autoimmune disorders may comprise anti-inflammatory drugs, corticosteroids, immunosuppressant drugs, or omega-3 derivatives, or a combination thereof.

### Systemic Conditions

Formulations applied to the periorbital or eyelid skin (NIODP) may be capable of delivering therapeutically adequate amounts of a compound to systemic circulation to treat all types of conditions effecting the body. In some embodiments, formulations may be delivered through NIODP to treat inflammatory soft tissue rheumatism, psoriatic arthritis, rheumatoid arthritis, and/or polyarthritis. Formulations delivered via NIODP to treat arthritis may comprise non-steroidal anti-inflammatory drugs (NSAIDs), corticosteroids, or disease-modifying antirheumatic drugs (DMARDs), or a combination thereof.

In some embodiments, formulations may be delivered via NIODP to treat ankylosing spondylitis. Formulations delivered via NIODP to treat ankylosing spondylitis may comprise non-steroidal anti-inflammatory drugs (NSAIDs), tofacitinib, etanercept, infliximab, adalimumab, golimumab, certolizumab, or secukinumab, or a combination thereof.

In some embodiments, formulations may be delivered via NIODP to treat gout. Formulations delivered via NIODP to treat gout may comprise non-steroidal anti-inflammatory drugs (NSAIDs), corticosteroids, allopurinol, febuxostat, probenecid, or pegloticase, or a combination thereof.

In some embodiments, formulations may be delivered via NIODP to treat thrombophlebitis. Formulations delivered via NIODP to treat thrombophlebitis may comprise non-steroidal anti-inflammatory drugs (NSAIDs), anticoagulants, or clot-dissolving medications (alteplase), or a combination thereof.

In some embodiments, formulations may be delivered via NIODP to treat vasculitis. Formulations delivered via NIODP to treat vasculitis may comprise corticosteroids, methotrexate, azathioprine, mycophenolate, cyclophosphamide, tocilizumab, or rituximab, or a combination thereof.

In some embodiments, formulations may be delivered via NIODP to treat renal fibrosis, chronic renal failure, and/or chronic liver diseases. Formulations delivered via NIODP to treat liver disease may comprise vitamin E, pioglitazone, or ursodiol, or a combination thereof.

In some embodiments, formulations may be delivered via NIODP to treat pleurisy. Formulations delivered via NIODP to treat pleurisy may comprise NSAIDs.

In some embodiments, formulations may be delivered via NIODP to treat gastrointestinal diseases such as colitis, irritable bowel syndrome, Crohn's disease, or diverticulitis. Formulations delivered via NIODP to treat gastrointestinal diseases may comprise infliximab, adalimumab, golimumab, alosetron, eluxadoline, rifaximin, lubiprostone, linaclotide, ciprofloxacin, metronidazole, trimethoprim-sulfamethoxazole, or moxifloxacin, or a combination thereof.

In some embodiments, formulations may be delivered via NIODP to treat endometriosis. Formulations delivered via NIODP to treat endometriosis may comprise elagolix or aromatase inhibitors, or a combination thereof.

In some embodiments, formulations may be delivered via NIODP to treat parasitic diseases. Formulations delivered via NIODP to treat parasitic diseases may comprise levamisole, niclosamide, praziquantel, albendazole, diethylcarbamazine, ivermectin, or tiabendazole, or a combination thereof.

In some embodiments, formulations may be delivered via NIODP to treat cancer. Formulations delivered via NIODP to treat cancer may comprise chemotherapy drugs, immunotherapy drugs, or hormone therapy, or a combination thereof.

### Cardiovascular Diseases

In some embodiments, formulations may be delivered via NIODP to treat cardiovascular related diseases including deep vein thrombosis, atherosclerosis, pulmonary fibrosis, hypertension, hypertriglyceridemia, type 2 diabetes, obesity, and atherothrombosis. Formulations delivered via NIODP to treat cardiovascular diseases may comprise anticoagulants including apixaban, dabigatran, edoxaban, heparin, rivaroxaban, or warfarin, or a combination thereof. Formulations delivered via NIODP to treat cardiovascular diseases may comprise antiplatelet agents and dual antiplatelet therapy (DAPTS) including aspirin, clopidogrel, dipyridamole, prasugrel, or ticagrelor, or a combination thereof. Formulations delivered via NIODP to treat cardiovascular diseases may comprise angiotensin-converting enzyme (ACE) inhibitors including benazepril, captopril, enalapril, fosinopril, lisinopril, moexipril, perindopril, quinapril, ramipril, or trandolapril, or a combination thereof. Formulations delivered via NIODP to treat cardiovascular diseases may comprise angiotensin II receptor blockers including azilsartan, candesartan, eprosartan, irbesartan, losartan, olmesartan, telmisartan, or valsartan, or a combination thereof. Formulations delivered via NIODP to treat cardiovascular diseases may comprise beta blockers including acebutolol, atenolol, betaxolol, bisoprolol, metoprolol, nadolol, propranolol, or sotalol, or a combination thereof. Formulations delivered via NIODP to treat cardiovascular diseases may comprise statins including atorvastatin, fluvastatin, lovastatin, pitavastatin, pravastatin, rosuvastatin, or simvastatin, or a combination thereof. Formulations delivered via NIODP to treat cardiovascular diseases may include diuretic or vasodilators.

### Respiratory Diseases

In some embodiments, formulations may be delivered via NIODP to treat respiratory diseases including allergies, asthma, lung inflammation, lung leukocyte infiltration, bronchial hyperresponsiveness, and allergic rhinitis. Formulations delivered via NIODP to treat respiratory diseases may comprise bronchodilators, nitric oxide, mucolytics, corticosteroids, leukotriene receptor antagonists, antihistamines, respiratory stimulants, pulmonary surfactants, or antiviral medications, or a combination thereof.

### Orthopedic Diseases

In some embodiments, formulations may be delivered via NIODP to treat orthopedic diseases including bone and cartilage destruction, osteoporosis, and osteoarthritis. Formulations delivered via NIODP to treat orthopedic diseases may comprise alendronate, risedronate, ibandronate, zoledronic acid, romosozumab, or a combination thereof.

### Dermatological Conditions

In some embodiments, formulations may be delivered via NIODP to treat dermatologic conditions including atopic dermatitis, polymyositis, dermatomyositis, erythema nodosum, nodular acne, prurigo nodularis, palmoplantar pustulosis, eczema, and psoriasis. Formulations delivered via NIODP to treat dermatologic conditions may comprise steroids, retinoids, biologics, methotrexate, or cyclosporine, or a combination thereof.

### Omega-3 Fatty Acids

The World Health Organization suggests that adults get 200 to 500 milligrams of omega-3 fatty acids, including docosahexaenoic acid (DHA) and eicosapentaenoic acid (EPA), per day for general health benefits. There are three main forms of omega-3 fatty acids: DHA and EPA, which are rich in cold-water fish oil, and alpha-linolenic acid (ALA), which is commonly derived from vegetable sources.

It is well known that Docosahexaenoic acid (DHA), an omega-3 fatty acid, plays important functional roles, such as anti-inflammation, anti-angiogenesis, antiapoptotic, and neuroprotection in brain and other systemic diseases. Studies in animal models suggest that above normal nutritional requirement intake of DHA might modify the risk/course of some diseases of the brain. Compared to other organs, DHA is highly enriched in the membrane phospholipids of the brain and neuronal tissue through intake of daily dietary nutrition.

Approximately 4 mg per day of DHA is metabolized in the human brain, and it undergoes a daily replenish and recycle processes during renewal of photoreceptor cells. The retina is an extension of the brain, so we would sometimes collectively call the brain and retina “the brain.” In the past, the pathway of DHA uptake from dietary nutrition absorption to systemic blood circulation, and then across of the blood-brain barrier (BBB) to the brain, has been extensively discussed. Essential fatty acids, such as DHA, other omega-3 polyunsaturated fatty acids (n-3 PUFAs), vitamin A, etc., have been proposed to cross the BBB through three potential mechanisms: passive diffusion, specialized transport proteins and transcytosis.

Additionally, omega-3 fatty acids, including DHA, may produce a local anesthetic effect. DHA has been shown to attenuate the nociceptive jaw-opening reflex in rats and may be a therapeutic agent and complementary alternative medicine for the prevention of acute trigeminal nociception or trigeminal neuralgia. DHA has been shown to demonstrate analgesic effects in post-operative pain after bone fracture in mice. DHA may promote the resolution of acute inflammation and inhibit inflammatory and neuropathic pain. DHA or its derivatives may be administered via NIODP to inhibit inflammatory and neuropathic pain. DHA or its derivatives may be administered via NIODP to treat, prevent, or reduce the severity of migraines. Omega-3 fatty acids may be administered to the periorbital skin or eyelid skin to reduce triglyceride levels in adult patients with moderate or severe hypertriglyceridemia. Omega-3 fatty acids may be administered to the periorbital skin or eyelid skin to prevent and treat retinopathy of prematurity.

However, DHA is among the most difficult to orally consume in sufficient amounts for ocular benefits because it is contained in few food sources. Thus, there exists a need for improved delivery methods of DHA to treat diseases of the brain, central nervous system, and the rest of the body.

In an aspect provided herein is a method of delivering one or more omega-3 fatty acids to the brain, central nervous system, and/or through systemic circulation to all parts of a body via periorbital or eyelid skin administration. The omega-3 fatty acids can be derived from any suitable source. In some embodiments, the omega-3 fatty acid is isolated from fish tissue. The concentration of omega-3 in fish oil may be increased through ethylation. In some embodiments, the omega-3 fatty acid is isolated from a plant source. In some embodiments, the plant source of omega-3 fatty acid is algae, seaweed, nori, spirulina, or chlorella. In some embodiments, the plant source of omega-3 fatty acid is flaxseed oil.

In some embodiments, the omega-3 fatty acid is a C16 to C24 omega-3 fatty acid, or a combination of C16 to C24 omega-3 fatty acids. In some embodiments, the omega-3 fatty acid is a C18 to C22 omega-3 fatty acid, or a combination of C18 to C22 omega-3 fatty acids.

In some embodiments, the omega-3 fatty acid is hexadecatrienoic acid (HTA), α-linolenic acid (ALA), stearidonic acid (SDA), eicosatrienoic acid (ETE), eicosatetraenoic acid (ETA), eicosapentaenoic acid (EPA), heneicosapentaenoic acid (HPA), docosapentaenoic acid (DPA), docosahexaenoic acid (DHA), tetracosapentaenoic acid, tetracosahexaenoic acid, or any combination thereof. In some embodiments, the omega-3 fatty acid comprises tetraconsenoic acid, hexacosenoic acid, octacosenoic acid, or any combination thereof. In some embodiments, the omega-3 fatty acid comprises ALA, EPA, DHA, or any combination thereof. In some embodiments, the omega-3 fatty acid comprises DHA. In some embodiments, the omega-3 fatty acid comprises EPA. In some embodiments, the omega-3 fatty acid comprises ALA. In some embodiments, the omega-3 fatty acid comprises both DHA and EPA.

In some embodiments, the omega-3 fatty acid is in the form of an omega-3 ethyl ester. Once in the skin, omega-3 ethyl esters can be converted by esterase to omega-3 free acid, which can easily pass the intercellular lipids of stratum corneum and hair pores. In some embodiments, the omega-3 fatty acid comprises a DHA ester. In some embodiments, the omega-3 fatty acid comprises an EPA ester. In some embodiments, the omega-3 fatty acid comprises a DHA ethyl ester. In some embodiments, the omega-3 fatty acid comprises an EPA ethyl ester. In some embodiments, the omega-3 fatty acid comprises an ester of both DHA and EPA. In some embodiments, the omega-3 fatty acid comprises an ethyl ester of both DHA and EPA. In some embodiments, the omega-3 fatty acid comprises omega-3-carboxylic acids (free fatty acids primarily composed of EPA and DHA). In some embodiments, the omega-3 fatty acid comprises icosapent ethyl (the ethyl ester of EPA).

In some embodiments, the omega-3 fatty acid is in the form of an omega-3 triglyceride. Natural fish oil contains the omega-3 fatty acids EPA and DHA mostly in the form of omega-3 triglycerides. Omega-3 triglycerides have a molecular weight around 900 Da. Once in the skin, omega-3 triglycerides can be converted by lipase to omega-3 free acid. Omega-3 triglycerides may also pass the intercellular lipids of stratum corneum and through hair pores.

In some embodiments, metabolites of omega-3 fatty acids may be administered to the periorbital or eyelid skin of the eye to provide substantial biodistribution in the tissues throughout the eye, brain, and systemically throughout the body. In some embodiments, the omega-3 fatty acid metabolite may comprise a leukotriene or a derivative thereof. In some embodiments, the omega-3 fatty acid may comprise a lipoxin or a derivative thereof. In some embodiments, the omega-3 fatty acid metabolite may comprise a 5-series leukotriene (LTB5, LTC5, LTD5, LTE5). In some embodiments, the omega-3 fatty acid metabolite may comprise a prostanoid, such as a prostacyclin, thromboxane, or prostaglandin, or a derivative thereof. In some embodiments, the omega-3 fatty acid metabolite may comprise a 3-series of prostanoids or prostaglandins. In some embodiments, the omega-3 fatty acid metabolite may comprise prostaglandin E3. In some embodiments, the omega-3 fatty acid metabolite may comprise prostaglandin 13. In some embodiments, the omega-3 fatty acid metabolite may comprise prostaglandin F3a. In some embodiments, the omega-3 fatty acid metabolite may comprise thromboxane A3. While EPA is great for helping lower chronic pain and inflammation anywhere in the body (for example: for cardiovascular health or diseases), DHA is best for the brain. To support brain health, the essential fatty acid supplement may have at least a ratio of 4:1 DHA to EPA. In some embodiments, the essential fatty acid supplement may have a ratio of 3:1 DHA to EPA. In some embodiments, the essential fatty acid supplement may have a ratio of 2:1 DHA to EPA. In some embodiments, the essential fatty acid supplement may have a ratio of 1:1 DHA to EPA. In some embodiments, the omega-3 fatty acid metabolite may comprise a maresin or a derivative thereof. In some embodiments, the omega-3 fatty acid metabolite may comprise a resolvin or a derivative thereof. In some embodiments, the omega-3 fatty acid metabolite may comprise a protectin or a derivative thereof.

Combinations with Omega-3 Fatty Acids

In treating various diseases or disorders, an omega-3 fatty acid or a pharmaceutically acceptable ester or salt thereof (DHA, EPA, etc.) can be combined with one or more additional therapeutic agents. An omega-3 fatty acid or a pharmaceutically acceptable ester or salt thereof can be combined with one or more additional therapeutics, and this combination can be administered periorbitally or topically to a subject's eyelid. An omega-3 fatty acid or a pharmaceutically acceptable ester or salt thereof can be combined with any of the therapeutics or active ingredients described herein. In some cases, the omega-3 fatty acid is cross-linked with any of the therapeutics or active ingredients described herein.

In some embodiments, a patient being treated with an omega-3 fatty acid or a pharmaceutically acceptable ester or salt thereof is further administered a VEGF antibody, or a functional fragment thereof.

In some embodiments, a patient being treated with an omega-3 fatty acid or a pharmaceutically acceptable ester or salt thereof is further administered a tyrosine kinase inhibitor.

In some embodiments, a patient being treated with an omega-3 fatty acid or a pharmaceutically acceptable ester or salt thereof is further administered a steroidal anti-inflammatory agent. In some embodiments, the steroidal anti-inflammatory again is selected from a group consisting of cortisone, prednisolone, methylprednisolone, raimcinolone, fluromethalone, medrysone, dexamethasone, lotprednol, hexacatonide, betamethasone, paramethasone, diflorasone, fluocinonide, fluocinolone, fluticasone, and triamcinolone.

In some embodiments, a patient being treated with an omega-3 fatty acid or a pharmaceutically acceptable ester or salt thereof is further administered a non-steroidal anti-inflammatory agent. In some embodiments, the non-steroidal anti-inflammatory agent is selected from a group consisting of ketorolac, nepafenac, amfenac, aspirin, indomethacin, flurbiprofen, ibuprofen, rofecoxib, and celecoxib.

In some embodiments, a patient being treated with an omega-3 fatty acid or a pharmaceutically acceptable ester or salt thereof is further administered an immunosuppressant. In some embodiments, the immunosuppressant is selected from a group consisting of cyclosporine, liftegrast, methotrexate, azathioprine, inhibitors of the PI3K-AKT-mTOR signaling pathway, (such as sirolimus, idelalisib, copanlisib, duvelisib, alpelisib, umbralisib, linperlisib, buparlisib, or BGB-10188), and agents that interfere with activation and function of the complement pathway (e.g. POT-4, ARC1905).

In some embodiments, the patient is co-administered an omega-3 fatty acid or a pharmaceutically acceptable ester or salt thereof and cyclosporine.

In some embodiments, the patient is co-administered an omega-3 fatty acid or a pharmaceutically acceptable ester or salt thereof and liftegrast.

In some embodiments, the patient is co-administered an omega-3 fatty acid or a pharmaceutically acceptable ester or salt thereof and methotrexate.

In some embodiments, the patient is co-administered an omega-3 fatty acid or a pharmaceutically acceptable ester or salt thereof and azathioprine. In some embodiments, the patient is co-administered an omega-3 fatty acid or a pharmaceutically acceptable ester or salt thereof and inhibitors of the PI3K-AKT-mTOR signaling pathway (such as sirolimus, idelalisib, copanlisib, duvelisib, alpelisib, umbralisib, linperlisib, buparlisib, or BGB-10188).

In some embodiments, a patient being treated with an omega-3 fatty acid or a pharmaceutically acceptable ester or salt thereof is further administered a nicotinic anti-cholinergic agent. In some embodiments, the nicotinic anti-cholinergic agent is selected from a group consisting of hexamethonium, decamethonium, and mecamyline.

In some embodiments, a patient being treated with an omega-3 fatty acid or a pharmaceutically acceptable ester or salt thereof is further administered thalidomide.

In some embodiments, a patient being treated with an omega-3 fatty acid or a pharmaceutically acceptable ester or salt thereof is further administered a prostaglandin receptor antagonist. In some embodiments, the antagonist blocks multiple prostaglandin receptors. In some embodiments, the antagonist is AGN 211377 and AGN 225660.

In some embodiments, a patient being treated with an omega-3 fatty acid or a pharmaceutically acceptable ester or salt thereof is further administered a neuroprotective agent. In some embodiments, the neuroprotective agent is selected from a group consisting of α₂-adrenoceptor agonists (e.g. brimonidine), NMDA antagonists (e.g. memantine), AMPA antagonists, Ca²⁺ blockers, σ-Irs-receptor agonists, pentazocine, endothelin receptor antagonists, Kinin antagonists, and anti-TNFα antibodies,

In some embodiments, a patient being treated with an omega-3 fatty acid or a pharmaceutically acceptable ester or salt thereof is further administered a neurotrophic/neuroregenerative agent (e.g ciliary neurotrophic factor, nerve growth factor, brain derived neurotrophic factor, 1 glial derived neurotrophic factor, neurotrophin 3), heat shock proteins, JNK inhibitors, synthetic bile acids (e.g. UDCA, TUDCA), progesterone, dopaminergics, neurotrophic factors, caspase inhibitors, acetyl-L-carnitine, acetylcholinesterase inhibitors, citicoline, acetylcysteine, retinoids (e.g. fenretinide), emixustat, anti-protein aggregation agents, phosphodiesterase inhibitors, nicotinamide, cannabinoids, citicholine, curcumin, minocycline, edaravone, erythropoietin, estrogen, L-theanine, melatonin, minocycline, noopept, pyrroloquinoline quinone, selegiline, simvastatin, esketamine, methylphenidate, ponesimod, glatiramer acetate, paliperidone, and vinpocetine agents that interferes with activation and function of the complement pathway, and vinpocetine.

In some embodiments, a patient being treated with an omega-3 fatty acid or a pharmaceutically acceptable ester or salt thereof is further administered an RNA interfering molecule. In some embodiments, the RNA interfering molecule may be siRNA, miRNA, or shRNA. In some embodiments the RNA interfering molecules is complementary to the gene sequence which encodes for a protein. In some embodiments, the RNA interfering molecule has a sequence that is at least partially complementary to the gene sequence, which encodes for a protein. In some embodiments, presence of the RNA interfering molecule produces silencing of the gene which encodes for a protein. In some embodiments the protein is a receptor. In some embodiments, a combination of at least two RNA interfering molecules are further administered to the patient. In some embodiments, at combination of at least two RNA interfering molecules silence the genes encoding for at least two proteins. In some embodiments the protein is an enzyme. In some embodiments the protein is selected from the group VEGF, PDGF, bFGF, SDF-1, HIF-1, PIGF, GLUT-1, Claudin cell adhesion molecules, HMBG-1, HuR, Ets1, GSK3β, RTP801, caspases 2-,3-,7-, PGC-1, ICAM1, t-PA, SNAI1, TBK1, ARMS2, TERT, ASK-1, and Nrf-2.

In some embodiments the RNA interfering molecule is a single stranded RNA. In some embodiments the RNA interfering molecule is a double stranded RNA. In some embodiments, the strand length of the RNA interfering molecule is about 10 nucleotides to about 200 nucleotides. In some embodiments, the strand length of the RNA interfering molecule is about 10 nucleotides to about 20 nucleotides, about 10 nucleotides to about 30 nucleotides, about 10 nucleotides to about 40 nucleotides, about 10 nucleotides to about 50 nucleotides, about 10 nucleotides to about 60 nucleotides, about 10 nucleotides to about 70 nucleotides, about 10 nucleotides to about 80 nucleotides, about 10 nucleotides to about 90 nucleotides, about 10 nucleotides to about 100 nucleotides, about 10 nucleotides to about 200 nucleotides, about 20 nucleotides to about 30 nucleotides, about 20 nucleotides to about 40 nucleotides, about 20 nucleotides to about 50 nucleotides, about 20 nucleotides to about 60 nucleotides, about 20 nucleotides to about 70 nucleotides, about 20 nucleotides to about 80 nucleotides, about 20 nucleotides to about 90 nucleotides, about 20 nucleotides to about 100 nucleotides, about 20 nucleotides to about 200 nucleotides, about 30 nucleotides to about 40 nucleotides, about 30 nucleotides to about 50 nucleotides, about 30 nucleotides to about 60 nucleotides, about 30 nucleotides to about 70 nucleotides, about 30 nucleotides to about 80 nucleotides, about 30 nucleotides to about 90 nucleotides, about 30 nucleotides to about 100 nucleotides, about 30 nucleotides to about 200 nucleotides, about 40 nucleotides to about 50 nucleotides, about 40 nucleotides to about 60 nucleotides, about 40 nucleotides to about 70 nucleotides, about 40 nucleotides to about 80 nucleotides, about 40 nucleotides to about 90 nucleotides, about 40 nucleotides to about 100 nucleotides, about 40 nucleotides to about 200 nucleotides, about 50 nucleotides to about 60 nucleotides, about 50 nucleotides to about 70 nucleotides, about 50 nucleotides to about 80 nucleotides, about 50 nucleotides to about 90 nucleotides, about 50 nucleotides to about 100 nucleotides, about 50 nucleotides to about 200 nucleotides, about 60 nucleotides to about 70 nucleotides, about 60 nucleotides to about 80 nucleotides, about 60 nucleotides to about 90 nucleotides, about 60 nucleotides to about 100 nucleotides, about 60 nucleotides to about 200 nucleotides, about 70 nucleotides to about 80 nucleotides, about 70 nucleotides to about 90 nucleotides, about 70 nucleotides to about 100 nucleotides, about 70 nucleotides to about 200 nucleotides, about 80 nucleotides to about 90 nucleotides, about 80 nucleotides to about 100 nucleotides, about 80 nucleotides to about 200 nucleotides, about 90 nucleotides to about 100 nucleotides, about 90 nucleotides to about 200 nucleotides, or about 100 nucleotides to about 200 nucleotides. In some embodiments, the strand length of the RNA interfering molecule is about 10 nucleotides, about 20 nucleotides, about 30 nucleotides, about 40 nucleotides, about 50 nucleotides, about 60 nucleotides, about 70 nucleotides, about 80 nucleotides, about 90 nucleotides, about 100 nucleotides, or about 200 nucleotides. In some embodiments, the strand length of the RNA interfering molecule is at least about 10 nucleotides, about 20 nucleotides, about 30 nucleotides, about 40 nucleotides, about 50 nucleotides, about 60 nucleotides, about 70 nucleotides, about 80 nucleotides, about 90 nucleotides, or about 100 nucleotides. In some embodiments, the strand length of the RNA interfering molecule is at most about 20 nucleotides, about 30 nucleotides, about 40 nucleotides, about 50 nucleotides, about 60 nucleotides, about 70 nucleotides, about 80 nucleotides, about 90 nucleotides, about 100 nucleotides, or about 200 nucleotides.

In some embodiments the RNA interfering molecules may prevent expression of VEGF receptors or attenuate the biosynthesis of VEGF and its various isoforms.

In some embodiments, a patient being treated with an omega-3 fatty acid or a pharmaceutically acceptable ester or salt thereof is further administered an RNA interfering molecule selected from a group that reduces or abolishes receptor expression or reduces the biosynthesis of PDGF, bFGF, SDF-1, HIF-1, PIGF, GLUT-1, Claudin cell adhesion molecules, HMBG-1, HuR, Ets1, GSK3B, RTP801, caspases 2-,3-,7-, PGC-1, ICAM1, t-PA, SNAI1, TBK1, SRPK1, C1Q, HtrA1, ARMS2, TERT, ASK-1, and Nrf-2.

In some embodiments, a patient being treated with an omega-3 fatty acid or a pharmaceutically acceptable ester or salt thereof is further administered an anti-oxidant. In some embodiments, the anti-oxidant is selected from a group consisting of β-carotene, lutein, zeaxanthin, riboflavin, Niacin, and polyunsaturated fatty acids such as docosohexanoic acid (DHA), eicosapentanoic acid (EPA), vitamin B₃, vitamin B₆, vitamin B₉, vitamin B₁₂, vitamin C, vitamin E, CoQ10, ghrelin, α-lipoic acid, resveratrol, flavinoids, gingko bilbao extract, ICAPS R®, OFTAN MACULA®, and epigallocatechin-3-gallate.

In some embodiments, a patient being treated with an omega-3 fatty acid or a pharmaceutically acceptable ester or salt thereof is further administered a therapeutic antibody. In some embodiments, the therapeutic antibody is a PDGF, FGF, PIGF, SDF-1, or HIF-1 antibody. In some embodiments, the therapeutic antibody is an antibody that interferes with activation and function of the complement pathway. In some embodiments, the administration of an omega-3 fatty acid or a pharmaceutically acceptable ester or salt thereof reduces the amount of the therapeutic antibody that would otherwise need to be administered to produce an intended therapeutic effect. In some embodiments, the reduced amount of therapeutic antibody is manifested as a lower dose of therapeutic antibody, or preferably, fewer or less frequent injections of the therapeutic antibody.

In some embodiments, a patient being treated with an omega-3 fatty acid or a pharmaceutically acceptable ester or salt thereof is further administered a therapeutic epigenetic modulator of acylating, deacylating, methylating, or demethylating histone proteins. In some embodiments, the omega-3 fatty acid or a pharmaceutically acceptable ester or salt thereof and the additional therapeutic agent are formulated together (e.g., as a single composition to be applied to the periorbital or eyelid skin of a patient).

In some embodiments, a patient being treated with an omega-3 fatty acid or a pharmaceutically acceptable ester or salt thereof is further administered chlorpromazine, haloperidol, amisulpiride, levomepromazine, norclozapine, olanzapine, paliperidone, quetiapine, risperidone, sulpiride, triapride, ziprasidone, thioridazine, aripiprazole, chlorpromazine, clozapine, flupentixol, melperone, perphenazine, pimozide, raclopride, thioridazine, thioxanthene, trifluperazine, hydrochloride, or ziprasidone, or a combination thereof.

In some embodiments, a patient being treated with an omega-3 fatty acid or a pharmaceutically acceptable ester or salt thereof is further administered C-Donepezil, Donepezil, Memantine, Rivastigmine, or Galantamine, or a combination thereof.

In some embodiments, a patient being treated with an omega-3 fatty acid or a pharmaceutically acceptable ester or salt thereof is further administered Efavirenz, Atazanavir, Emtricitabine, Lamivudine, Lopinavir, or Tenofovir, or a combination thereof.

### Other Active Ingredients and Excipients

In some embodiments, the compound or mixture suitable for topical periorbital or eyelid administration is selected from the group consisting of the following families of compounds: C—C motif receptor 3 (CCR3) inhibitors, vitamin A and modified forms of vitamin A (such as NCT03845582), complement factor 1q inhibitors, Apurinic/Apyrimidinic Endonuclease 1/Redox Effector Factor-1 (APE1/Ref-1) inhibitors, Steroids, endothelial cell (EC)-specific receptor tyrosine kinases Tie2 agonists (activators), angiopoietin-2 antagonists, Retinol-binding protein 4 (RBP4) antagonists, Complement component 3 (C3) inhibitors, pan-arginylglycylaspartic acid (RGD) integrin antagonists, connexin43 hemichannels blockers, Complement component 5 inhibitors, pan RGD integrin antagonists, Rho kinase inhibitors, Ref-1 inhibitors, AP endonuclease 1 inhibitors, serine/threonine-protein kinase (SRPK1) inhibitors, CC3 (TIP30=tat-interacting protein of 30 kDa) inhibitors, Complement C1Q Inhibitors, Compliment factor (B, and D) inhibitors, C3 convertase inhibitors, C5 convertase inhibitors, Inflammasome inhibitors, HtrA1 inhibitors, Matrix modulators (such as doxycycline), MASP2 (MBL Associated Serine Protease 2) blockers, MASP3 (MBL Associated Serine Protease 3) blockers, Antiviral drugs (such as Ganciclovir), VEGF receptor (R1, R2, R3) inhibitors, PDGF receptor inhibitors (including imatinib, sorafenib, and sunitinib), Prostanoid IP receptor antagonists (for example: RO3244794), tyrosine kinases inhibitors, PAF (platelet-activating factor) receptor inhibitors, and combinations of two or more thereof.

In some embodiments, a pharmaceutical composition suitable for topical periorbital or eyelid administration may comprise any of the following families of vitamins and antioxidants: vitamin A (Retinol) and modified forms of vitamin A (such as retinoic acid, retinoids e.g. retinyl acetate, retinyl linoleate, retinyl palmitate and retinyl proprionate, carotenoids, β-carotene, and NCT03845582); vitamin B3 encompassing nicotinamide (also known as niacinamide), nicotinic acid (also known as niacin) and nicotinamide riboside; the derivatives of vitamin B3, such as nicotine, nicotinamide adenine dinucleotide (NAD+), NADAH, NADP+, and NADPH; The vitamin C (L-Ascorbic Acid) and its derivatives, such as sodium ascorbyl phosphate, magnesium ascorbyl phosphate, ascorbyl glucoside, tetrahexyldecyl ascorbate and ethylated L-ascorbic acid.

In some embodiments, a pharmaceutical composition suitable for topical periorbital or eyelid administration may comprise an anti-oxidant. In some embodiments, the anti-oxidant is selected from a group consisting of lutein, zeaxanthin, riboflavin, and polyunsaturated fatty acids such as docosohexanoic acid (DHA), eicosapentanoic acid (EPA), vitamin B₃, vitamin B₆, vitamin B₉, vitamin B₁₂, vitamin C, vitamin E, CoQ10, ghrelin, α-lipoic acid, resveratrol, flavinoids, gingko bilbao extract, ICAPS R®, OFTAN MACULA®, and epigallocatechin-3-gallate.

In some embodiments, a pharmaceutical composition suitable for topical periorbital or eyelid administration may comprise an additional therapeutic agent. In some embodiments, the additional therapeutic agent is a VEGF antibody, a PDGF antibody, a bFGF antibody, a SDF-1 antibody, a HIF-1 antibody, a PIGF antibody, a VEGF antagonist, a tyrosine kinase inhibitor, a steroidal anti-inflammatory agent, a non-steroidal anti-inflammatory agent, an immunosuppressant, an anti-cholinergic agent, thalidomide, a prostaglandin receptor antagonist, a neuroprotective agent, a neuro-regenerative agent, an RNA interference molecule that provides neuroprotection, an RNA interference molecule that promotes neuro-regeneration, a small molecule that directly provides neuroprotection and reduces intraocular pressure, an RNA interference molecule that promotes neuro-regeneration and reduces intraocular pressure, an RNA interference that provides neuroprotection and reduces intraocular pressure, an antibody that reduces edema, hemorrhage, and angiogenesis, an RNA interference that reduces edema, hemorrhage and angiogenesis.

In some embodiments, a pharmaceutical composition suitable for topical periorbital or eyelid administration may comprise a ligand (including endogenous and exogenous agonists, antagonists, inhibitors) of the endocannabinoid system. Since some ligands (including endogenous and exogenous agonists, antagonists, inhibitors) of the endocannabinoid system are lipophilic and hard to deliver to the brain, delivery to the periorbital or eyelid skin may be the most appropriate route of delivery to reduce adverse effects and increase the efficacy of cannabinoid treatment in targeted tissues, particularly in the brain/CNS. In some cases, ligands of the endocannabinoid system may be salts. In some cases, ligands of the endocannabinoid system may be polar. The endogenous cannabinoids (endocannabinoids) can include anandamide (AEA), 2-arachidonoylglycerol (2-AG), arachidonic acid (AA), 2-arachidonyl-glycero-lether (noladin, 2-AGE), O-arachidonyl-ethanolamine (viro-dhamine), and N-arachidonoyl-dopamine (NADA). The endocannabinoid molecules may be endogenously synthesized from omega-3 and omega-6 polyunsaturated fatty acids (PUFA). Ligands of the endocannaboid system can include natural cannabis in marijuana, including delta (9)-tetrahydrocannabinol (delta (9)-THC, psychoactive), cannabidiol, and/or Dronabinol. Ligands of the endocannaboid system can include synthetic cannabinoid receptor agonists, including ajulemic acid, Dronabinol, Marinol, Nabilone, CP 55940, GW-100, HU-210, WIN 55212-2, JWH-015, Arachidonyl-2′-chloroethylamide (ACEA, CB1 agonist), Dexanabinol (HU-211, CB1 agonist), and/or HU-308 (CB2 agonist). Ligands of the endocannaboid system may include CB receptor antagonists. Ligands of the endocannaboid system may include CB1 receptor antagonists, including LH-21, LY320135, SR141716 (Rimonabant). Ligands of the endocannaboid system may include CB2 receptor antagonists, including AM 630 (and partial CB1 agonist) and/or SR145528. Other compounds that act via endocannabinoid receptor pathways include cannabinoid transport inhibitors such as AM 404 and VCM 707. Other compounds that act via endocannabinoid receptor pathways include FAAH-Resistant Transport Inhibitors such as AM 1172.

In one aspect, provided herein, a pharmaceutical composition suitable for topical periorbital or eyelid administration may comprise any pharmaceutically acceptable excipient. In some embodiments, the pharmaceutically acceptable excipient comprises one or more acids, bases, electrolytes, buffers, solutes, antioxidants, stabilizers, and if required, preservatives. In some embodiments, the pharmaceutically acceptable excipient comprises a semifluorinated alkane. In some embodiments, the pharmaceutically acceptable excipient comprises perfluorohexyloctane. In some embodiments, the pharmaceutically acceptable excipient comprises perfluorobutylpentane. In some embodiments, a pharmaceutical composition suitable for topical periorbital or eyelid administration comprises an omega-3 fatty acid or a pharmaceutically acceptable ester or salt thereof and a semifluorinated alkane. In some embodiments, a pharmaceutical composition suitable for topical periorbital or eyelid administration comprises an omega-3 fatty acid or a pharmaceutically acceptable ester or salt thereof and perfluorohexyloctane. In some embodiments, a pharmaceutical composition suitable for topical periorbital or eyelid administration comprises an omega-3 fatty acid or a pharmaceutically acceptable ester or salt thereof and perfluorobutylpentane.

In some embodiments, a composition for periorbital or eyelid delivery comprises an antibody. In some embodiments, the composition has a molecular weight of around 1000 kDa. In some embodiments, the composition has a molecular weight of around 150 kDa. In some embodiments, the composition has a molecular weight of about 7 kDa. In some embodiments, a composition for periorbital or eyelid delivery comprises a double stranded siRNA. In some embodiments, a composition for periorbital or eyelid delivery comprises a single stranded siRNA. In some embodiments, the composition comprises a short oligo peptide. In some embodiments, a composition for periorbital or eyelid delivery comprises a short DNA oligo nucleotides.

In some embodiments, a composition for periorbital or eyelid delivery comprises a viral vector. In some embodiments, the composition has a molecular weight of around 1000 kDa. In some embodiments, the composition comprises a RNA viral vector. In some embodiments, the composition comprises a DNA viral vector.

In some embodiments, the short oligo peptide is a sequence of about 1 amino acid to about 6 amino acids. In some embodiments, the short oligo peptide is a sequence of about 1 amino acid to about 2 amino acids, about 1 amino acid to about 3 amino acids, about 1 amino acid to about 4 amino acids, about 1 amino acid to about 5 amino acids, about 1 amino acid to about 6 amino acids, about 2 amino acids to about 3 amino acids, about 2 amino acids to about 4 amino acids, about 2 amino acids to about 5 amino acids, about 2 amino acids to about 6 amino acids, about 3 amino acids to about 4 amino acids, about 3 amino acids to about 5 amino acids, about 3 amino acids to about 6 amino acids, about 4 amino acids to about 5 amino acids, about 4 amino acids to about 6 amino acids, or about 5 amino acids to about 6 amino acids. In some embodiments, the short oligo peptide is a sequence of about 1 amino acid, about 2 amino acids, about 3 amino acids, about 4 amino acids, about 5 amino acids, or about 6 amino acids. In some embodiments, the short oligo peptide is a sequence of at least about 1 amino acid, about 2 amino acids, about 3 amino acids, about 4 amino acids, or about 5 amino acids. In some embodiments, the short oligo peptide is a sequence of at most about 2 amino acids, about 3 amino acids, about 4 amino acids, about 5 amino acids, or about 6 amino acids.

In some embodiments, a composition for periorbital or eyelid delivery comprises liposomes. In some embodiments the composition comprises nanoparticles. In some embodiments, the average diameter of the liposomes or nanoparticles is about 0.1 μm to about 1 μm. In some embodiments, the average diameter of the liposomes or nanoparticles is about 0.1 μm to about 0.2 μm, about 0.1 μm to about 0.3 μm, about 0.1 μm to about 0.4 μm, about 0.1 μm to about 0.5 μm, about 0.1 μm to about 0.6 μm, about 0.1 μm to about 0.7 μm, about 0.1 μm to about 0.8 μm, about 0.1 μm to about 0.9 μm, about 0.1 μm to about 1 μm, about 0.2 μm to about 0.3 μm, about 0.2 μm to about 0.4 μm, about 0.2 μm to about 0.5 μm, about 0.2 μm to about 0.6 μm, about 0.2 μm to about 0.7 μm, about 0.2 μm to about 0.8 μm, about 0.2 μm to about 0.9 μm, about 0.2 μm to about 1 μm, about 0.3 μm to about 0.4 μm, about 0.3 μm to about 0.5 μm, about 0.3 μm to about 0.6 μm, about 0.3 μm to about 0.7 μm, about 0.3 μm to about 0.8 μm, about 0.3 μm to about 0.9 μm, about 0.3 μm to about 1 μm, about 0.4 μm to about 0.5 μm, about 0.4 μm to about 0.6 μm, about 0.4 μm to about 0.7 μm, about 0.4 μm to about 0.8 μm, about 0.4 μm to about 0.9 μm, about 0.4 μm to about 1 μm, about 0.5 μm to about 0.6 μm, about 0.5 μm to about 0.7 μm, about 0.5 μm to about 0.8 μm, about 0.5 μm to about 0.9 μm, about 0.5 μm to about 1 μm, about 0.6 μm to about 0.7 μm, about 0.6 μm to about 0.8 μm, about 0.6 μm to about 0.9 μm, about 0.6 μm to about 1 μm, about 0.7 μm to about 0.8 μm, about 0.7 μm to about 0.9 μm, about 0.7 μm to about 1 μm, about 0.8 μm to about 0.9 μm, about 0.8 μm to about 1 μm, or about 0.9 μm to about 1 μm. In some embodiments, the average diameter of the liposomes or nanoparticles is about 0.1 μm, about 0.2 μm, about 0.3 μm, about 0.4 μm, about 0.5 μm, about 0.6 μm, about 0.7 μm, about 0.8 μm, about 0.9 μm, or about 1 μm. In some embodiments, the average diameter of the liposomes or nanoparticles is at least about 0.1 μm, about 0.2 μm, about 0.3 μm, about 0.4 μm, about 0.5 μm, about 0.6 μm, about 0.7 μm, about 0.8 μm, or about 0.9 μm. In some embodiments, the average diameter of the liposomes or nanoparticles is at most about 0.2 μm, about 0.3 μm, about 0.4 μm, about 0.5 μm, about 0.6 μm, about 0.7 μm, about 0.8 μm, about 0.9 μm, or about 1 μm.

In some embodiments, a composition for periorbital or eyelid delivery comprises a lipophilic compound. In some embodiments, the composition comprises a nonpolar compound. In some embodiments, the composition comprises a bipolar compound. In some embodiments, the composition comprises a zwitterion.

### NIODP for Particle Delivery

Because particles, including macromolecules and drugs, may be effectively delivered to the brain and systemically throughout the body via periorbital or eyelid administration, NIODP may be used as a particle delivery system. In some embodiments, drug delivery may involve nanoparticles. These may be selected from a group comprising polymeric, lipid based, liposomes, albumin bound, inorganic, organic crystals, and viral based nanoparticles

In an aspect provided herein is a method of delivering one or more particles to the brain, central nervous system, and/or through systemic circulation to all parts of a body via periorbital or eyelid skin administration. Particles may comprise macromolecules, such as peptides, proteins, oligonucleotides (RNA or DNA), nucleic acids (RNA and DNA), and viral vectors for gene therapy. Particles may also include low molecular weight drugs. Macromolecules can be packed in nanocarriers to form nanoparticles, such as liposomes, polymeric micelles, lipoplexes and polyplexes. A wide variety of active agents, varying from low molecular weight drug molecules to macromolecules such as proteins and nucleic acids, can be incorporated into nanoparticles. Liposomes have versatile properties in terms of size, surface charge and lipid composition. Liposomes have an ability to incorporate almost any drug independent of its solubility in water. Lipids may be used to deliver drugs or particles via NIODP. A large variety of lipids may be employable as a carrier for the preparation of liposomes. In some cases, mixtures of stearic acid and Tween 80 can be used. In some cases, mixtures of distearoyl phosphatidylcholine, distearoyl phosphatidylglycerol, and cholesterol, and/or diplasmenylcholine are used. In some cases, mixtures of distearoyl phosphatidylcholine, dimyristoyl phosphatidylglycerol, and cholesterol are used. In some cases, mixtures of castor oil, phosphatidylcholine and polyethylene glycol coupled to distearoylphosphatidylethanolamine are used.

In some cases, delivery may be facilitated or enhanced by increasing periorbital or eyelid skin penetration. Skin penetration may be enhanced by encompassing a particle in a material that is readily accepted and absorbed into the skin. In some cases, delivery may be facilitated by encompassing the particle in a carrier. A carrier can have the ability to rea may be a certain region of the body such as the gastrointestinal tract, cardiovascular system, reproductive system, respiratory system, or endocrine system. Mixtures of omega-3 fatty acids (DHA alone or with different composition of other omega-3 fatty acids) and their derivatives may be used as a safe carrier of particles. The particles may be incorporated into or complexed with active agents varying from low molecular weight drug molecules to macromolecules.

In addition to a particle delivery system, NIODP may be used to deliver foreign objects to the body. Foreign objects may comprise, for example, nanochips or biosensors. In some cases, delivery may be facilitated or enhanced by increasing periorbital or eyelid skin penetration. Skin penetration may be enhanced by encompassing a foreign object in a material that is readily accepted and absorbed into the skin. In some cases, delivery may be facilitated by encompassing the foreign object in a carrier. In some cases, a carrier may not release the foreign object until it has had a chance to reach its intended target. A carrier may have a high stability and long residence or circulate time after administration. In some cases, this ensures that a foreign object does not break down or deactivate preemptively. A carrier may have the capability to actively or passively deliver a foreign object to a target area. In some cases, the target area is the brain or central nervous system. The target area may be a certain region of the body such as the gastrointestinal tract, cardiovascular system, reproductive system, respiratory system, or endocrine system. In some cases, foreign objects are covered in a coating that is chosen specifically to mimic the chemistry of an intended target area. This may help facilitate and integrate the foreign object into the intended region of the body.

Mixtures of omega-3 fatty acids (DHA alone or with different composition of other omega-3 fatty acids) and their derivatives may be used as a carrier of foreign objects. Mixtures of omega-3 fatty acids (DHA alone or with different composition of other omega-3 fatty acids) and their derivatives may be used as a safe carrier of foreign object to be delivered via NIODP for CNS delivery, or delivery to any other area in the body with or without systemic circulation. In some cases, mixtures of omega-3 fatty acids (DHA alone or with different composition of other omega-3 fatty acids) may mimic the surface chemistry of an individual neuron, which can help facilitate and integrate the foreign object into the nervous system.

### Formulations

### Compositions for Periorbital or Eyelid Skin Administration

Provided herein in some embodiments are compositions suitable for application to the periorbital or eyelid skin region of the eye of a subject. In some embodiments, these compositions may be administered through a non-invasive ocular delivery platform (NIODP).

In some embodiments, the composition is in the form of an aqueous solution, a non-aqueous solution, an oil solution, an oil, a gel, a hydrogel, a lotion, an ointment, a dispersion, an emulsion, a cream, and a suspension. In some embodiments, the composition is in the form of an ointment, a cream, or a lotion. In some embodiments, the composition is in the form of an ointment. In some embodiments, the composition is in the form of an aqueous solution. In some embodiments, the composition is in the form of a non-aqueous solution. In some embodiments, the composition is in the form of an oil solution. In some embodiments, the composition is in the form of an oil. In some embodiments, the composition is in the form of a gel. In some embodiments, the composition is in the form of a hydrogel. In some embodiments, the composition is in the form of a lotion. In some embodiments, the composition is in the form of an ointment. In some embodiments, the composition is in the form of a dispersion. In some embodiments, the composition is in the form of an emulsion. In some embodiments, the composition is in the form of a cream. In some embodiments, the composition is in the form of a suspension.

In some embodiments, the composition comprises a semi-solid oleaginous base material. In some embodiments, the composition comprises a petroleum base, a mineral oil, a polyol, a triglyceride, or any combination thereof. In some embodiments, the composition comprises a petroleum base. In some embodiments, the composition comprises petrolatum. In some embodiments, the composition comprises petrolatum, a triglyceride, or any combination thereof.

In some embodiments, the composition comprises petrolatum and a triglyceride. In some embodiments, the composition comprises petrolatum, beeswax, or cocoa butter. In some embodiments, the composition comprises beeswax. In some embodiments, the composition comprises cocoa butter.

In some embodiments, the composition comprises an oil. In some embodiments, the composition comprises an oil or a mixture of oils. In some embodiments, the composition comprises a compound provided herein (e.g., an omega-3 fatty acid) mixed in one or more oils. In some embodiments, the composition comprises one or more oils derived from plants, plant seeds, or nuts. In some embodiments, the plant, plant seed, or nut is soybean, sesame, olive, vegetable, sunflower, or other plant source, or any combination thereof.

In some embodiments, the composition is mostly an oil. In some embodiments, the composition comprises an oil in an amount of at least about 90%, at least about 91%, at least about 92%, at least about 93%, at least about 94%, at least about 95%, at least about 96%, at least about 97%, at least about 98%, at least about 99%, at least about 99.5%, at least about 99.6%, at least about 99.7%, at least about 99.8%, at least about 99.9%, at least about 99.95%, at least about 99.96%, at least about 99.97%, at least about 99.98%, or at least about 99.99% (w/w) of the composition. In some embodiments, the composition consists essentially of the oil and an active ingredient. In some embodiments, the composition of the oil consists essentially of the active ingredient.

In some embodiments, the composition comprises the oil in an amount of about 1% to about 100% (w/w) of the composition. In some embodiments, the composition comprises the oil in an amount of about 1% to about 20%, about 1% to about 40%, about 1% to about 60%, about 1% to about 80%, about 1% to about 100%, about 20% to about 40%, about 20% to about 60%, about 20% to about 80%, about 20% to about 100%, about 40% to about 60%, about 40% to about 80%, about 40% to about 100%, about 60% to about 80%, about 60% to about 100%, or about 80% to about 100% (w/w) of the composition. In some embodiments, the composition comprises the oil in an amount of about 1%, about 20%, about 40%, about 60%, about 80%, or about 100% (w/w) of the composition. In some embodiments, the composition comprises the oil in an amount of at least about 1%, about 20%, about 40%, about 60%, or about 80% (w/w) of the composition. In some embodiments, the composition comprises the oil in an amount of at most about 20%, about 40%, about 60%, about 80%, or about 100% (w/w) of the composition.

In some embodiments, the composition comprises a triglyceride. In some embodiments, the triglyceride is a medium-chain or a long-chain triglyceride. In some embodiments, the triglyceride is derived from a natural source. In some embodiments, the triglyceride is derived from plants, plant seeds, or nuts. In some embodiments, the plant, plant seed, or nut comprises a part of a soybean, a sesame seed or plant, an olive, a sunflower seed or plant, or other vegetable or plant source, or any combination thereof.

In some embodiments, the composition is mostly a triglyceride. In some embodiments, the composition comprises a triglyceride in an amount of at least about 90%, at least about 91%, at least about 92%, at least about 93%, at least about 94%, at least about 95%, at least about 96%, at least about 97%, at least about 98%, at least about 99%, at least about 99.5%, at least about 99.6%, at least about 99.7%, at least about 99.8%, at least about 99.9%, at least about 99.95%, at least about 99.96%, at least about 99.97%, at least about 99.98%, or at least about 99.99% (w/w) of the composition. In some embodiments, the composition consists essentially of the triglyceride and an active ingredient.

In some embodiments, the composition comprises the triglyceride in an amount of about 1% to about 100% (w/w) of the composition. In some embodiments, the composition comprises the medium-chain triglyceride in an amount of about 1% to about 20%, about 1% to about 40%, about 1% to about 60%, about 1% to about 80%, about 1% to about 100%, about 20% to about 40%, about 20% to about 60%, about 20% to about 80%, about 20% to about 100%, about 40% to about 60%, about 40% to about 80%, about 40% to about 100%, about 60% to about 80%, about 60% to about 100%, or about 80% to about 100% (w/w) of the composition. In some embodiments, the composition comprises the triglyceride in an amount of about 1%, about 20%, about 40%, about 60%, about 80%, or about 100% (w/w) of the composition. In some embodiments, the composition comprises the triglyceride in an amount of at least about 1%, about 20%, about 40%, about 60%, or about 80% (w/w) of the composition. In some embodiments, the composition comprises the triglyceride in an amount of at most about 20%, about 40%, about 60%, about 80%, about 90%, or about 100% (w/w) of the composition.

In some embodiments, the triglyceride is a medium-chain triglyceride. In some embodiments, the medium-chain triglyceride comprises 2 or 3 medium length fatty acids. In some embodiments, the medium-chain triglyceride comprises C6 or larger fatty acids. In some embodiments, the medium chain triglyceride comprises C6 to C12 fatty acids. In some embodiments, the medium-chain triglyceride comprises a mixture of C6 to C12 fatty acids. In some embodiments, the medium-chain triglyceride comprises fatty acids selected from C6, C8, C10, and C12 fatty acids, or a mixture thereof. In some embodiments, the medium-chain triglyceride comprises caproic acid, caprylic acid, capric acid, lauric acid, or any combination thereof. In some embodiments, the medium-chain triglyceride comprises caprylic acid, capric acid, or a combination thereof. In some embodiments, the medium-chain triglyceride comprises caprylic acid and capric acid. In some embodiments, the medium-chain triglyceride comprises caprylic acid and capric acid in a ratio of about 4:1 (w/w), about 4:3 (w/w), about 3:1 (w/w), about 3:2 (w/w), about 1:1 (w/w), about 2:3 (w/w), about 1:3 (w/w), about 3:4 (w/w), or about 1:4 (w/w). In some embodiments, the ratio is from about 1:1 (w/w) to about 4:1 (w/w). In some embodiments, the ratio is about 3:2 (w/w). In some embodiments, the medium-chain triglyceride comprises at least 75%, at least 80%, at least 85%, at least 90%, at least 91%, at least 92%, at least 93%, at least 94%, or at least 95% C6 to C12 fatty acids as compared to other fatty acids (w/w).

In some embodiments, the composition is mostly a medium chain triglyceride. In some embodiments, the composition comprises a medium-chain triglyceride in an amount of at least about 90%, at least about 91%, at least about 92%, at least about 93%, at least about 94%, at least about 95%, at least about 96%, at least about 97%, at least about 98%, at least about 99%, at least about 99.5%, at least about 99.6%, at least about 99.7%, at least about 99.8%, at least about 99.9%, at least about 99.95%, at least about 99.96%, at least about 99.97%, at least about 99.98%, or at least about 99.99% (w/w) of the composition. In some embodiments, the composition consists essentially of the medium-chain triglyceride and an active ingredient

In some embodiments, the composition comprises the medium-chain triglyceride in an amount of about 1% to about 100% (w/w) of the composition. In some embodiments, the composition comprises the medium-chain triglyceride in an amount of about 1% to about 20%, about 1% to about 40%, about 1% to about 60%, about 1% to about 80%, about 1% to about 100%, about 20% to about 40%, about 20% to about 60%, about 20% to about 80%, about 20% to about 100%, about 40% to about 60%, about 40% to about 80%, about 40% to about 100%, about 60% to about 80%, about 60% to about 100%, or about 80% to about 100% (w/w) of the composition. In some embodiments, the composition comprises the medium-chain triglyceride in an amount of about 1%, about 20%, about 40%, about 60%, about 80%, or about 100% (w/w) of the composition. In some embodiments, the composition comprises the medium-chain triglyceride in an amount of at least about 1%, about 20%, about 40%, about 60%, or about 80% (w/w) of the composition. In some embodiments, the composition comprises the medium-chain triglyceride in an amount of at most about 20%, about 40%, about 60%, about 80%, or about 100% (w/w) of the composition.

In some embodiments, the medium-chain triglyceride is derived from a natural source. In some embodiments, the medium-chain triglyceride is derived from coconut, palm, or palm kernel, or combinations thereof. In some embodiments, the medium-chain triglyceride is derived from coconut, or palm. In some embodiments, the medium-chain triglyceride is the oil extracted from the endosperm of coconut or palm. In some embodiments, the medium-chain triglyceride is National Food (NF) grade (NF) or US Pharmacopeia (USP) grade.

In some embodiments, the composition comprises a mixture of petrolatum and a medium-chain triglyceride. In some embodiments, the ratio of petrolatum to medium-chain triglyceride is from about 10:1 (v/v) to about 1:2 (v/v). In some embodiments, the ratio of petrolatum to medium-chain triglyceride is from about 6:1 (v/v) to about 1:1 (v/v). In some embodiments, the ratio of petrolatum to medium-chain triglyceride is from about 6:1 (v/v) to about 1:1 (v/v), from about 5:1 (v/v) to about 1:1 (v/v), from about 4:1 (v/v) to about 1:1 (v/v), from about 3:1 (v/v) to about 2:1 (v/v), or from about 3:2 (v/v) to about 1:1 (v/v). In some embodiments, the ratio of petrolatum to medium-chain triglyceride is about 1:1 (v/v), about 2:1 (v/v), about 3:1 (v/v), about 4:1 (v/v), about 5:1 (v/v), or about 6:1 (v/v). In some embodiments, the ratio of petrolatum to medium-chain triglyceride is about 1:1 (v/v). In some embodiments, the ratio of petrolatum to medium-chain triglyceride is about 2:1 (v/v). In some embodiments, the ratio of petrolatum to medium-chain triglyceride is about 4:1 (v/v).

In some embodiments, the composition further comprises an emollient. In some embodiments, the emollient is selected from a group consisting of vegetable oils, mineral oils, essential oils, essential fatty acids, fatty acids, fatty acid esters, and fatty acid alcohols.

In some embodiments, the composition further comprises a humectant. In some embodiments, the humectant is selected from a group consisting of propylene glycol, aloe vera, lactic acid, glyceryl triacetate, lithium chloride, polydextrose, quillaia, sodium hexametaphosphate, glycerol, sorbitol, xylitol, maltitol, and castor oil.

In some embodiments, the composition further comprises a thickening agent. In some embodiments, the thickening agent is selected from a group consisting of fatty acids, fatty acid esters, and fatty acid alcohols.

In some embodiments, the composition further comprises a preservative. In some embodiments, the preservative is selected from a group consisting of sodium borate/boric acid, polyhexamthethylene biguanide (phmb), parabens (parahydroxy benzoic acid derivatives; phenyl mercuric nitrate, benzalkonium chloride, benzelthonium chloride, chlorhexidine, chlorbutanol, methyl paraben, phenylethyl alcohol, phenylmercuric acetate, phenylmercuric nitrate, propyl paraben, and thimerosal.

In some embodiments, the composition is free from preservatives. In some embodiments, the composition is free from benzalkonium chloride.

In some embodiments, the composition further comprises an antimicrobial. In some embodiments, the antimicrobial is selected from a group consisting of basil, oregano, thyme, citrus oils and monoterpene, sesquiterpenes, and phenylpropanoids.

In some embodiments, the composition further comprises a penetration enhancer. In some embodiments, the penetration enhancer is selected from a group consisting of ethanol, isopropyl alcohol, d-hexanol, octanol, doctanol, myristyl alcohol, ethyl acetate, oleoyl acetate, isopropyl myristate, azone, carbamide, glycerylmono-oleate, octyl salicylate, propylene glycol, dipropylene glycol, 1,2-butylene glycol, oleic acid, N-methyl-2-pyrrolidone, 2-pyrrolidone, 2-pyrrolidone-5-carboxylic acid, dimethyl sulfoxide, decylmethyl sulfoxide, sodium lauryl sulfate, Span 80, Tween 80, cineole, eugenol, D-limonene, menthol, menthane, cyclodextrins, hyaluronic acid, and vitamin E.

In some embodiments, the composition further comprises an odor masking agent. Odor masking agents are especially suitable for compositions which comprise a component derived from the tissue of an animal (e.g., omega-3 fatty acids derived from fish) which may carry a residual odor. In some embodiments, the odor masking agent is an essential oil (e.g., a floral, fruit, wood, mint, herbal, or other essential oil).

### Compositions of Omega-3 Fatty Acids for External Eyelid or Periorbital Skin Administration

In some embodiments, the compositions suitable for topical periorbital or eyelid skin administration provided herein comprise an omega-3 fatty acid, an omega-3 acid ethyl ester, an omega-3-carboxylic acid, or a pharmaceutically acceptable salt thereof, and a pharmaceutically acceptable excipient. In some embodiments, these compositions may be administered through a non-invasive ocular delivery platform (NIODP).

In some embodiments, the compositions suitable for topical periorbital or eyelid skin administration provided herein comprises an ester, carboxylic acid. In some embodiments, the compositions suitable for topical external eyelid skin administration provided herein comprise an omega-3 fatty acid, or a pharmaceutically acceptable salt thereof, and a pharmaceutically acceptable excipient.

In some embodiments, the omega-3 fatty acid is present in the composition of from about 0.01% to about 100% (w/w) of the composition. In some embodiments, the omega-3 fatty acid is present in an amount of about 0.01% to about 50% (w/w) of the composition. In some embodiments, the omega-3 fatty acid is present in an amount of about 1% to about 50% (w/w) of the composition. In some embodiments, the omega-3 fatty acid is present in an amount of about 1% to about 5%, about 1% to about 10%, about 1% to about 20%, about 1% to about 30%, about 1% to about 40%, about 1% to about 50%, about 5% to about 10%, about 5% to about 20%, about 5% to about 30%, about 5% to about 40%, about 5% to about 50%, about 5% to about 75%, about 5% to about 100%, about 10% to about 20%, about 10% to about 30%, about 10% to about 40%, about 10% to about 50%, about 10% to about 75%, about 10% to about 100%, about 20% to about 30%, about 20% to about 40%, about 20% to about 50%, about 20% to about 75%, about 20% to about 100%, about 30% to about 40%, about 30% to about 50%, about 30% to about 75%, about 30% to about 100%, about 40% to about 50%, about 40% to about 75%, about 40% to about 100%, about 50% to about 75%, about 50% to about 100%, or about 75% to about 100%, (w/w) of the composition. In some embodiments, the omega-3 fatty acid is present in an amount of up to about 20%, about 30%, about 40%, about 50%, about 60%, about 70%, about 80%, about 90% or about 100% (w/w) of the composition. In some embodiments, the omega-3 fatty acid is present in an amount of about 10% to about 15%, about 10% to about 20%, about 10% to about 25%, about 10% to about 30%, about 15% to about 20%, about 15% to about 25%, about 15% to about 30%, about 20% to about 25%, about 20% to about 30%, or about 25% to about 30% (w/w) of the composition. In some embodiments, the omega-3 fatty acid is present in an amount of about 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, or 100% (w/w) of the composition. In some embodiments, the omega-3 fatty acid is administered alone (e.g., without any vehicle).

In some embodiments, the composition is configured to deliver the omega-3 fatty acid in an amount of about 0.01 mg to about 3000 mg, 0.01 mg to about 1000 mg, about 0.01 mg to about 500 mg, about 0.01 mg to about 200 mg, about 0.01 mg to about 100 mg, about 0.01 mg to about 50 mg, about 0.01 mg to about 20 mg, about 0.01 mg to about 10 mg, about 0.01 mg to about 5 mg, about 0.01 mg to about 2 mg, about 0.01 mg to about 1 mg, about 0.01 mg to about 0.5 mg, or about 0.01 mg to about 0.1 mg. In some embodiments, the composition is configured to deliver the omega-3 fatty acid in an amount of about 0.1 mg to about 100 mg. In some embodiments, the composition is configured to deliver the omega-3 fatty acid in an amount of about 0.1 mg to about 1 mg, about 0.1 mg to about 10 mg, about 0.1 mg to about 20 mg, about 0.1 mg to about 50 mg, about 0.1 mg to about 100 mg, about 1 mg to about 10 mg, about 1 mg to about 20 mg, about 1 mg to about 50 mg, about 1 mg to about 100 mg, about 10 mg to about 20 mg, about 10 mg to about 50 mg, about 10 mg to about 100 mg, about 20 mg to about 50 mg, about 20 mg to about 100 mg, or about 50 mg to about 100 mg. In some embodiments, the composition is configured to deliver the omega-3 fatty acid in an amount of about 0.1 mg, about 1 mg, about 10 mg, about 20 mg, about 50 mg, or about 100 mg. In some embodiments, the composition is configured to deliver the omega-3 fatty acid in an amount of at least about 0.1 mg, about 1 mg, about 10 mg, about 20 mg, or about 50 mg. In some embodiments, the composition is configured to deliver the omega-3 fatty acid in an amount of at most about 1 mg, about 10 mg, about 20 mg, about 50 mg, or about 100 mg

In some embodiments, the composition comprises a vehicle for the delivery of the omega-3 fatty acid. In some embodiments, the vehicle comprises an oil. In some embodiments, the vehicle comprises an oil or a mixture of oils. In some embodiments, the vehicle comprises an omega-3 fatty acid dissolved in one or more oils. In some embodiments, the oil is derived from a natural source. In some embodiments, the vehicle comprises one or more oils derived from plants, plant seeds, or nuts. In some embodiments, the plant, plant seed, or nut is soybean, sesame, olive, vegetable, sunflower, or other plant source, or any combination thereof.

In some embodiments, the vehicle is an oil. In some embodiments, the composition comprises the oil in an amount of about 1% to about 100% (w/w) of the composition. In some embodiments, the composition comprises the oil in an amount of about 1% to about 20%, about 1% to about 40%, about 1% to about 60%, about 1% to about 80%, about 1% to about 100%, about 20% to about 40%, about 20% to about 60%, about 20% to about 80%, about 20% to about 100%, about 40% to about 60%, about 40% to about 80%, about 40% to about 100%, about 60% to about 80%, about 60% to about 100%, or about 80% to about 100% (w/w) of the composition. In some embodiments, the composition comprises the oil in an amount of about 1%, about 20%, about 40%, about 60%, about 80%, or about 100% (w/w) of the composition. In some embodiments, the composition comprises the oil in an amount of at least about 1%, about 20%, about 40%, about 60%, or about 80% (w/w) of the composition. In some embodiments, the composition comprises the oil in an amount of at most about 20%, about 40%, about 60%, about 80%, or about 100% (w/w) of the composition. In some embodiments, the oil comprises from about 50% to about 99% (w/w) of the composition. In some embodiments, the oil comprises about 50% to about 60%, about 50% to about 70%, about 50% to about 80%, about 50% to about 90%, about 50% to about 99%, about 60% to about 70%, about 60% to about 80%, about 60% to about 90%, about 60% to about 99%, about 70% to about 80%, about 70% to about 90%, about 70% to about 99%, about 80% to about 90%, about 80% to about 99%, or about 90% to about 99% (w/w) of the composition. In some embodiments, the oil comprises about 50%, about 60%, about 70%, about 80%, about 90%, or about 99% (w/w) of the composition.

In some embodiments, the vehicle comprises a triglyceride. In some embodiments, the triglyceride is a medium-chain or a long-chain triglyceride. In some embodiments, the triglyceride is derived from a natural source. In some embodiments, the triglyceride is derived from plants, plant seeds, or nuts. In some embodiments, the plant, plant seed, or nut comprises a part of a soybean, a sesame seed or plant, an olive, a sunflower seed or plant, or other vegetable or plant source, or any combination thereof.

In some embodiments, the composition comprises the triglyceride in an amount of about 1% to about 100% (w/w) of the composition. In some embodiments, the composition comprises the medium-chain triglyceride in an amount of about 1% to about 20%, about 1% to about 40%, about 1% to about 60%, about 1% to about 80%, about 1% to about 100%, about 20% to about 40%, about 20% to about 60%, about 20% to about 80%, about 20% to about 100%, about 40% to about 60%, about 40% to about 80%, about 40% to about 100%, about 60% to about 80%, about 60% to about 100%, or about 80% to about 100% (w/w) of the composition. In some embodiments, the composition comprises the triglyceride in an amount of about 1%, about 20%, about 40%, about 60%, about 80%, or about 100% (w/w) of the composition. In some embodiments, the composition comprises the triglyceride in an amount of at least about 1%, about 20%, about 40%, about 60%, or about 80% (w/w) of the composition. In some embodiments, the composition comprises the triglyceride in an amount of at most about 20%, about 40%, about 60%, about 80%, or about 100% (w/w) of the composition. In some embodiments, the triglyceride comprises from about 50% to about 99% (w/w) of the composition. In some embodiments, the triglyceride comprises about 50% to about 60%, about 50% to about 70%, about 50% to about 80%, about 50% to about 90%, about 50% to about 99%, about 60% to about 70%, about 60% to about 80%, about 60% to about 90%, about 60% to about 99%, about 70% to about 80%, about 70% to about 90%, about 70% to about 99%, about 80% to about 90%, about 80% to about 99%, or about 90% to about 99% (w/w) of the composition. In some embodiments, the triglyceride comprises about 50%, about 60%, about 70%, about 80%, about 90%, or about 99% (w/w) of the composition.

In some embodiments, the triglyceride is a medium-chain triglyceride. In some embodiments, the medium-chain triglyceride comprises 2 or 3 medium length fatty acids. In some embodiments, the medium-chain triglyceride comprises C6 or larger fatty acids. In some embodiments, the medium chain triglyceride comprises C6 to C12 fatty acids. In some embodiments, the medium-chain triglyceride comprises a mixture of C6 to C12 fatty acids. In some embodiments, the medium-chain triglyceride comprises fatty acids selected from C6, C8, C10, and C12 fatty acids, or a mixture thereof. In some embodiments, the medium-chain triglyceride comprises caproic acid, caprylic acid, capric acid, lauric acid, or any combination thereof. In some embodiments, the medium-chain triglyceride comprises caprylic acid, capric acid, or a combination thereof. In some embodiments, the medium-chain triglyceride comprises caprylic acid and capric acid. In some embodiments, the medium-chain triglyceride comprises caprylic acid and capric acid in a ratio of about 4:1 (w/w), about 4:3 (w/w), about 3:1 (w/w), about 3:2 (w/w), about 1:1 (w/w), about 2:3 (w/w), about 1:3 (w/w), about 3:4 (w/w), or about 1:4 (w/w). In some embodiments, the ratio is from about 1:1 (w/w) to about 4:1 (w/w). In some embodiments, the ratio is about 3:2 (w/w). In some embodiments, the medium-chain triglyceride comprises at least 75%, at least 80%, at least 85%, at least 90%, at least 91%, at least 92%, at least 93%, at least 94%, or at least 95% C6 to C12 fatty acids as compared to other fatty acids (w/w).

In some embodiments, the composition comprises the medium-chain triglyceride in an amount of about 1% to about 100% (w/w) of the composition. In some embodiments, the composition comprises the medium-chain triglyceride in an amount of about 1% to about 20%, about 1% to about 40%, about 1% to about 60%, about 1% to about 80%, about 1% to about 100%, about 20% to about 40%, about 20% to about 60%, about 20% to about 80%, about 20% to about 100%, about 40% to about 60%, about 40% to about 80%, about 40% to about 100%, about 60% to about 80%, about 60% to about 100%, or about 80% to about 100% (w/w) of the composition. In some embodiments, the composition comprises the medium-chain triglyceride in an amount of about 1%, about 20%, about 40%, about 60%, about 80%, or about 100% (w/w) of the composition. In some embodiments, the composition comprises the medium-chain triglyceride in an amount of at least about 1%, about 20%, about 40%, about 60%, or about 80% (w/w) of the composition. In some embodiments, the composition comprises the medium-chain triglyceride in an amount of at most about 20%, about 40%, about 60%, about 80%, or about 100% (w/w) of the composition. In some embodiments, the medium-chain triglyceride comprises from about 50% to about 99% (w/w) of the composition. In some embodiments, the medium-chain triglyceride comprises about 50% to about 60%, about 50% to about 70%, about 50% to about 80%, about 50% to about 90%, about 50% to about 99%, about 60% to about 70%, about 60% to about 80%, about 60% to about 90%, about 60% to about 99%, about 70% to about 80%, about 70% to about 90%, about 70% to about 99%, about 80% to about 90%, about 80% to about 99%, or about 90% to about 99% (w/w) of the composition. In some embodiments, the medium-chain triglyceride comprises about 50%, about 60%, about 70%, about 80%, about 90%, or about 99% (w/w) of the composition.

In some embodiments, the medium-chain triglyceride is derived from a natural source. In some embodiments, the medium-chain triglyceride is derived from coconut, palm, or palm kernel, or combinations thereof. In some embodiments, the medium-chain triglyceride is derived from coconut, or palm. In some embodiments, the medium-chain triglyceride is the oil extracted from the endosperm of coconut or palm. In some embodiments, the medium-chain triglyceride is National Food (NF) grade (NF) or US Pharmacopeia (USP) grade.

In some embodiments, the vehicle is a fatty acid vehicle. In some embodiments, the fatty acid vehicle is an unsaturated fatty acid. In some embodiments, the fatty acid vehicle is a C14 to C22 fatty acid. In some embodiments, the fatty acid vehicle is a C14 to C22 unsaturated fatty acid. In some embodiments, the fatty acid vehicle comprises linoleic acid.

In some embodiments, the fatty acid vehicle comprises from about 1% to about 100% (w/w) of the composition. In some embodiments, the fatty acid vehicle comprises from about 1% to about 20%, about 1% to about 40%, about 1% to about 60%, about 1% to about 80%, about 1% to about 100%, about 20% to about 40%, about 20% to about 60%, about 20% to about 80%, about 20% to about 100%, about 40% to about 60%, about 40% to about 80%, about 40% to about 100%, about 60% to about 80%, about 60% to about 100%, or about 80% to about 100% (w/w) of the composition. In some embodiments, the fatty acid vehicle comprises from about 1%, about 20%, about 40%, about 60%, about 80%, or about 100% (w/w) of the composition. In some embodiments, the fatty acid vehicle comprises from at least about 1%, about 20%, about 40%, about 60%, or about 80% (w/w) of the composition. In some embodiments, the fatty acid vehicle comprises from at most about 20%, about 40%, about 60%, about 80%, or about 100% (w/w) of the composition. In some embodiments, the fatty acid vehicle comprises from about 50% to about 99% (w/w) of the composition. In some embodiments, the fatty acid vehicle comprises about 50% to about 60%, about 50% to about 70%, about 50% to about 80%, about 50% to about 90%, about 50% to about 99%, about 60% to about 70%, about 60% to about 80%, about 60% to about 90%, about 60% to about 99%, about 70% to about 80%, about 70% to about 90%, about 70% to about 99%, about 80% to about 90%, about 80% to about 99%, or about 90% to about 99% (w/w) of the composition. In some embodiments, the fatty acid vehicle comprises about 50%, about 60%, about 70%, about 80%, about 90%, or about 99% (w/w) of the composition.

In some embodiments, administration of the composition to a patient via NIODP results in a physiologically relevant amount of the omega-3 fatty acid to at least one portion of the brain. In some embodiments, administration of the composition to a patient via NIODP results in a physiologically relevant amount of the omega-3 fatty acid to at least one portion of the central nervous system. In some embodiments, administration of the composition to a patient via NIODP results in a physiologically relevant amount of the omega-3 fatty acid circulated systemically throughout the body of a subject.

In some embodiments, administration of the composition to a patient via NIODP results in a amount of omega-3 fatty acid in the brain sufficient to prevent, treat, or slow/stop disease progression in the brain. In some cases, administration of the composition to a patient via NIODP results in a therapeutically or other beneficially relevant amount of the omega-3 fatty acid to at least one portion of the brain. In some cases, a condition of the brain is prevented or treated, symptoms associated therewith are reduced, or brain deterioration is stopped after periorbital or eyelid administration of a topical composition comprising about 100 micrograms to about 500,000 micrograms of omega-3 fatty acid. In some cases, a condition of the brain is prevented or treated, symptoms associated therewith are reduced, or brain deterioration is stopped after periorbital or eyelid administration of a topical composition comprising about 100 micrograms to about 500 micrograms, about 100 micrograms to about 1,000 micrograms, about 100 micrograms to about 10,000 micrograms, about 100 micrograms to about 100,000 micrograms, about 100 micrograms to about 500,000 micrograms, about 500 micrograms to about 1,000 micrograms, about 500 micrograms to about 10,000 micrograms, about 500 micrograms to about 100,000 micrograms, about 500 micrograms to about 500,000 micrograms, about 1,000 micrograms to about 10,000 micrograms, about 1,000 micrograms to about 100,000 micrograms, about 1,000 micrograms to about 500,000 micrograms, about 10,000 micrograms to about 100,000 micrograms, about 10,000 micrograms to about 500,000 micrograms, or about 100,000 micrograms to about 500,000 micrograms of omega-3 fatty acid. In some cases, a condition of the brain is prevented or treated, symptoms associated therewith are reduced, or brain deterioration is stopped after periorbital or eyelid administration of a topical composition comprising about 100 micrograms, about 500 micrograms, about 1,000 micrograms, about 10,000 micrograms, about 100,000 micrograms, or about 500,000 micrograms of omega-3 fatty acid. In some cases, a condition of the brain is prevented or treated, symptoms associated therewith are reduced, or brain deterioration is stopped after periorbital or eyelid administration of a topical composition comprising at least about 100 micrograms, about 500 micrograms, about 1,000 micrograms, about 10,000 micrograms, or about 100,000 micrograms of omega-3 fatty acid. In some cases, a condition of the brain is prevented or treated, symptoms associated therewith are reduced, or brain deterioration is stopped after periorbital or eyelid administration of a topical composition comprising at most about 500 micrograms, about 1,000 micrograms, about 10,000 micrograms, about 100,000 micrograms, or about 500,000 micrograms of omega-3 fatty acid.

In some embodiments, administration of the composition to a patient via a non-invasive ocular delivery platform results in a therapeutically or other beneficially relevant amount of the omega-3 fatty acid to at least one portion of the central nervous system. In some embodiments, administration of the composition to a patient via a non-invasive ocular delivery platform results in a therapeutically or other beneficially relevant amount of the omega-3 fatty acid circulated systemically throughout the body of a subject.

### Methods of Treatment

Provided herein are methods of treatment of any disease and disorder provided herein with a compound as provided herein. In some embodiments, the compound is an omega-3 fatty acid.

In some embodiments, the compound is administered to the periorbital or eyelid skin of each eye of the patient four times per day, three times per day, twice per day, once per day, once every other day, once every three days, once every four days, or once every seven days, or any combination thereof (e.g. a variable dosing protocol). In some embodiments, the compound is administered to the periorbital or eyelid skin of each eye of the patient once per week, twice per week, three times per week, once every two weeks, or once every three weeks. In some embodiments, the compound is administered to the periorbital or eyelid skin of each eye of the patient once per day.

In some embodiments, the compound is administered to the periorbital or eyelid skin of one eye of the patient twice per day, once per day, once every other day, once every three days, once every four days, or once every seven days, or any combination thereof (e.g. a variable dosing protocol). In some embodiments, the compound is administered to the periorbital or eyelid skin of one eye of the patient once per week, twice per week, three times per week, once a week, once every two weeks, or once every three weeks. In some embodiments, the compound is administered to the periorbital or eyelid skin of one eye of the patient once per day.

In some embodiments, the compound is administered ad libitum with respect to the periorbital or eyelid skin of either or both eyes.

In some embodiments, the compound is applied to the periorbital or eyelid skin using a device. In some embodiments, the device is a dropper, a pump, a spray, a click pen or a tube reservoir device. In some embodiments, the compound is administered topically by brush, Q-tip, spatula, or a fingertip.

In some embodiments, the device releases a preselected dosage in a uniform manner onto the periorbital or eyelid skin of the patient. In some embodiments, the compound is applied by a roller device to the periorbital or eyelid skin. In some embodiments, the compound is applied by a Q-tip to the periorbital or eyelid skin. In some embodiments, the compound is applied by a spatula to the periorbital or eyelid skin. In some embodiments, the compound is applied by a fingertip to the periorbital or eyelid skin. In some embodiments, the application process may be preceded by using a graduated dropper, syringe, click pen or pipette.

In some embodiments, the compound is applied to the periorbital or eyelid skin using an eye pad. An eye pad, also known as eye patch, is a small (and may be sterile) pad large enough to cover the periorbital or eyelid region of the eye, specifically designed for absorption of formulation for periorbital or eyelid administration. In some embodiments, the eye pad comprises a preselected dosage of an active ingredient. A subject may apply the eye pad to the periorbital or eyelid skin for a certain period of time. The time may depend on the desired dose of active ingredient desired. In some embodiments, the eye pad may be applied to the periorbital or eyelid skin of a patient for 1 minute, 5 minutes, 10 minutes, 20 minutes, 30 minutes, 45 minutes, or 1 hour.

It may be beneficial to use formulation that does not comprise preservatives. In some embodiments, the compound is packaged in a single-use container. In some embodiments, the single-use container is a blow-fill-seal capsule. In some embodiments, the single-use container is a soft gel capsule. In some embodiments, the compound is packaged in a multi-use container. In some embodiments, the multi-use container is an airless pump or drop bottle. In some embodiments, packaging is designed to minimize the fishy smell that may be caused my oxidation of an omega-3 fatty acid.

In some embodiments, the compound is administered to the periorbital skin above the upper eyelid, below the lower eyelid, or both above the upper and below the lower eyelids. In some embodiments, the compound is administered above the upper eyelid. In some embodiments, the compound is administered below the lower eyelid. In some embodiments, the compound is administered both above the upper and below the lower eyelid.

In some embodiments, the compound is administered to the upper eyelid, the lower eyelid, or both the upper and the lower eyelids. In some embodiments, the compound is administered on the upper eyelid. In some embodiments, the compound is administered on the lower eyelid. In some embodiments, the compound is administered on both the upper and the lower eyelid.

In some embodiments, penetration through the periorbital or eyelid skin is increased by a penetration enhancer, tape-stripping, microdermabrasion, solvent, pulsed laser, and iontophoresis, which has been found useful for delivering macromolecules e.g. antibodies, siRNAs, in liposomes or in nanoparticles.

In some embodiments, the compound is applied to the eyelid skin using a device. In some embodiments, the device is a dropper, a pump, a spray, a click pen or a tube reservoir device. In some embodiments, the compound is administered topically by a brush, Q-tip, spatula, or fingertip.

In some embodiments, the device releases a preselected dosage in a uniform manner onto the eyelid skin of the patient. In some embodiments, the compound is applied by a roller device to the eyelid skin. In some embodiments, the compound is applied by a Q-tip to the eyelid skin. In some embodiments, the compound is applied by a spatula to the eyelid skin. In some embodiments, the compound is applied by a fingertip to the eyelid skin. In some embodiments, the application process may be preceded by using a graduated dropper, syringe, click pen or pipette.

In some embodiments, penetration through the eyelid skin is increased by a penetration enhancer, tape-stripping, microdermabrasion, solvent, pulsed laser, and iontophoresis, which has been found useful for delivering macromolecules e.g. antibodies, siRNAs, in liposomes or in nanoparticles (Fukuta et al. (2020) J Control Release 10:323-332).

In some embodiments, the compound is administered prophylactically, as an emergency intervention, or as required to achieve the desired remedial effects.

In some cases, the percentage of omega-3 fatty acid delivered to the brain after administration is about 0.04% to about 50% of the administered dose. In some cases, the percentage of omega-3 fatty acid delivered to the brain after administration is about 0.04% to about 0.05%, 0.05% to about 0.1%, about 0.05% to about 0.5%, about 0.05% to about 1%, about 0.05% to about 3%, about 0.05% to about 5%, about 0.05% to about 10%, about 0.05% to about 50%, about 0.1% to about 0.5%, about 0.1% to about 1%, about 0.1% to about 3%, about 0.1% to about 5%, about 0.1% to about 10%, about 0.1% to about 50%, about 0.5% to about 1%, about 0.5% to about 3%, about 0.5% to about 5%, about 0.5% to about 10%, about 0.5% to about 50%, about 1% to about 3%, about 1% to about 5%, about 1% to about 10%, about 1% to about 50%, about 3% to about 5%, about 3% to about 10%, about 3% to about 50%, about 5% to about 10%, about 5% to about 50%, or about 10% to about 50%. In some cases, the percentage of omega-3 fatty acid delivered to the brain after administration is about 0.04%, 0.05%, about 0.1%, about 0.5%, about 1%, about 3%, about 5%, about 10%, or about 50%. In some cases, the percentage of omega-3 fatty acid delivered to the brain after administration is at least about 0.04%, 0.05%, about 0.1%, about 0.5%, about 1%, about 3%, about 5%, or about 10%. In some cases, the percentage of omega-3 fatty acid delivered to the brain after administration is at most about 0.1%, about 0.5%, about 1%, about 3%, about 5%, about 10%, or about 50% of the administered dose.

In some cases, after administration of an omega-3 fatty acid to the periorbital or eyelid skin of a subject, the level of omega-3 fatty acid in the subject's brain increases by about 0.5% to about 300%. In some cases, after administration of an omega-3 fatty acid to the periorbital or eyelid skin of a subject, the level of omega-3 fatty acid in the subject's brain increases by about 0.5% to about 1%, about 0.5% to about 5%, about 0.5% to about 25%, about 0.5% to about 50%, about 0.5% to about 100%, about 0.5% to about 300%, about 1% to about 5%, about 1% to about 25%, about 1% to about 50%, about 1% to about 100%, about 1% to about 300%, about 5% to about 25%, about 5% to about 50%, about 5% to about 100%, about 5% to about 300%, about 25% to about 50%, about 25% to about 100%, about 25% to about 300%, about 50% to about 100%, about 50% to about 300%, or about 100% to about 300%. In some cases, after administration of an omega-3 fatty acid to the periorbital or eyelid skin of a subject, the level of omega-3 fatty acid in the subject's brain increases by about 0.5%, about 1%, about 5%, about 25%, about 50%, about 100%, or about 300%. In some cases, after administration of an omega-3 fatty acid to the periorbital or eyelid skin of a subject, the level of omega-3 fatty acid in the subject's brain increases by at least about 0.5%, about 1%, about 5%, about 25%, about 50%, or about 100%. In some cases, after administration of an omega-3 fatty acid to the periorbital or eyelid skin of a subject, the level of omega-3 fatty acid in the subject's brain increases by at most about 1%, about 5%, about 25%, about 50%, about 100%, or about 300%.

In some cases, after administration of an omega-3 fatty acid to the periorbital or eyelid skin of a subject, the level of omega-3 fatty acid in the subject's brain is about 60 nmol/g of tissue to about 300 nmol/g of tissue. In some cases, after administration of an omega-3 fatty acid to the periorbital or eyelid skin of a subject, the level of omega-3 fatty acid in the subject's brain is about 60 nmol/g of tissue to about 75 nmol/g of tissue, about 60 nmol/g of tissue to about 90 nmol/g of tissue, about 60 nmol/g of tissue to about 100 nmol/g of tissue, about 60 nmol/g of tissue to about 110 nmol/g of tissue, about 60 nmol/g of tissue to about 120 nmol/g of tissue, about 60 nmol/g of tissue to about 150 nmol/g of tissue, about 60 nmol/g of tissue to about 200 nmol/g of tissue, about 60 nmol/g of tissue to about 300 nmol/g of tissue, about 75 nmol/g of tissue to about 90 nmol/g of tissue, about 75 nmol/g of tissue to about 100 nmol/g of tissue, about 75 nmol/g of tissue to about 110 nmol/g of tissue, about 75 nmol/g of tissue to about 120 nmol/g of tissue, about 75 nmol/g of tissue to about 150 nmol/g of tissue, about 75 nmol/g of tissue to about 200 nmol/g of tissue, about 75 nmol/g of tissue to about 300 nmol/g of tissue, about 90 nmol/g of tissue to about 100 nmol/g of tissue, about 90 nmol/g of tissue to about 110 nmol/g of tissue, about 90 nmol/g of tissue to about 120 nmol/g of tissue, about 90 nmol/g of tissue to about 150 nmol/g of tissue, about 90 nmol/g of tissue to about 200 nmol/g of tissue, about 90 nmol/g of tissue to about 300 nmol/g of tissue, about 100 nmol/g of tissue to about 110 nmol/g of tissue, about 100 nmol/g of tissue to about 120 nmol/g of tissue, about 100 nmol/g of tissue to about 150 nmol/g of tissue, about 100 nmol/g of tissue to about 200 nmol/g of tissue, about 100 nmol/g of tissue to about 300 nmol/g of tissue, about 110 nmol/g of tissue to about 120 nmol/g of tissue, about 110 nmol/g of tissue to about 150 nmol/g of tissue, about 110 nmol/g of tissue to about 200 nmol/g of tissue, about 110 nmol/g of tissue to about 300 nmol/g of tissue, about 120 nmol/g of tissue to about 150 nmol/g of tissue, about 120 nmol/g of tissue to about 200 nmol/g of tissue, about 120 nmol/g of tissue to about 300 nmol/g of tissue, about 150 nmol/g of tissue to about 200 nmol/g of tissue, about 150 nmol/g of tissue to about 300 nmol/g of tissue, or about 200 nmol/g of tissue to about 300 nmol/g of tissue. In some cases, after administration of an omega-3 fatty acid to the periorbital or eyelid skin of a subject, the level of omega-3 fatty acid in the subject's brain is about 60 nmol/g of tissue, about 75 nmol/g of tissue, about 90 nmol/g of tissue, about 100 nmol/g of tissue, about 110 nmol/g of tissue, about 120 nmol/g of tissue, about 150 nmol/g of tissue, about 200 nmol/g of tissue, or about 300 nmol/g of tissue. In some cases, after administration of an omega-3 fatty acid to the periorbital or eyelid skin of a subject, the level of omega-3 fatty acid in the subject's brain is at least about 60 nmol/g of tissue, about 75 nmol/g of tissue, about 90 nmol/g of tissue, about 100 nmol/g of tissue, about 110 nmol/g of tissue, about 120 nmol/g of tissue, about 150 nmol/g of tissue, or about 200 nmol/g of tissue. In some cases, after administration of an omega-3 fatty acid to the periorbital or eyelid skin of a subject, the level of omega-3 fatty acid in the subject's brain is at most about 75 nmol/g of tissue, about 90 nmol/g of tissue, about 100 nmol/g of tissue, about 110 nmol/g of tissue, about 120 nmol/g of tissue, about 150 nmol/g of tissue, about 200 nmol/g of tissue, or about 300 nmol/g of tissue.

In some cases, after administration of an omega-3 fatty acid to the periorbital or eyelid skin of a subject, the level of DHA in the subject's brain is about 60 nmol/g of tissue to about 300 nmol/g of tissue. In some cases, after administration of an omega-3 fatty acid to the periorbital or eyelid skin of a subject, the level of DHA in the subject's brain is about 60 nmol/g of tissue to about 75 nmol/g of tissue, about 60 nmol/g of tissue to about 90 nmol/g of tissue, about 60 nmol/g of tissue to about 100 nmol/g of tissue, about 60 nmol/g of tissue to about 110 nmol/g of tissue, about 60 nmol/g of tissue to about 120 nmol/g of tissue, about 60 nmol/g of tissue to about 150 nmol/g of tissue, about 60 nmol/g of tissue to about 200 nmol/g of tissue, about 60 nmol/g of tissue to about 300 nmol/g of tissue, about 75 nmol/g of tissue to about 90 nmol/g of tissue, about 75 nmol/g of tissue to about 100 nmol/g of tissue, about 75 nmol/g of tissue to about 110 nmol/g of tissue, about 75 nmol/g of tissue to about 120 nmol/g of tissue, about 75 nmol/g of tissue to about 150 nmol/g of tissue, about 75 nmol/g of tissue to about 200 nmol/g of tissue, about 75 nmol/g of tissue to about 300 nmol/g of tissue, about 90 nmol/g of tissue to about 100 nmol/g of tissue, about 90 nmol/g of tissue to about 110 nmol/g of tissue, about 90 nmol/g of tissue to about 120 nmol/g of tissue, about 90 nmol/g of tissue to about 150 nmol/g of tissue, about 90 nmol/g of tissue to about 200 nmol/g of tissue, about 90 nmol/g of tissue to about 300 nmol/g of tissue, about 100 nmol/g of tissue to about 110 nmol/g of tissue, about 100 nmol/g of tissue to about 120 nmol/g of tissue, about 100 nmol/g of tissue to about 150 nmol/g of tissue, about 100 nmol/g of tissue to about 200 nmol/g of tissue, about 100 nmol/g of tissue to about 300 nmol/g of tissue, about 110 nmol/g of tissue to about 120 nmol/g of tissue, about 110 nmol/g of tissue to about 150 nmol/g of tissue, about 110 nmol/g of tissue to about 200 nmol/g of tissue, about 110 nmol/g of tissue to about 300 nmol/g of tissue, about 120 nmol/g of tissue to about 150 nmol/g of tissue, about 120 nmol/g of tissue to about 200 nmol/g of tissue, about 120 nmol/g of tissue to about 300 nmol/g of tissue, about 150 nmol/g of tissue to about 200 nmol/g of tissue, about 150 nmol/g of tissue to about 300 nmol/g of tissue, or about 200 nmol/g of tissue to about 300 nmol/g of tissue. In some cases, after administration of an omega-3 fatty acid to the periorbital or eyelid skin of a subject, the level of DHA in the subject's brain is about 60 nmol/g of tissue, about 75 nmol/g of tissue, about 90 nmol/g of tissue, about 100 nmol/g of tissue, about 110 nmol/g of tissue, about 120 nmol/g of tissue, about 150 nmol/g of tissue, about 200 nmol/g of tissue, or about 300 nmol/g of tissue. In some cases, after administration of an omega-3 fatty acid to the periorbital or eyelid skin of a subject, the level of DHA in the subject's brain is at least about 60 nmol/g of tissue, about 75 nmol/g of tissue, about 90 nmol/g of tissue, about 100 nmol/g of tissue, about 110 nmol/g of tissue, about 120 nmol/g of tissue, about 150 nmol/g of tissue, or about 200 nmol/g of tissue. In some cases, after administration of an omega-3 fatty acid to the periorbital or eyelid skin of a subject, the level of DHA in the subject's brain is at most about 75 nmol/g of tissue, about 90 nmol/g of tissue, about 100 nmol/g of tissue, about 110 nmol/g of tissue, about 120 nmol/g of tissue, about 150 nmol/g of tissue, about 200 nmol/g of tissue, or about 300 nmol/g of tissue.

After administration of an omega-3 fatty acid to the periorbital or eyelid skin of a subject, a target concentration of omega-3 fatty acid in a subject's brain may be maintained post-dosage for a given time period. In some cases, a target concentration of omega-3 fatty acid is maintained for one hour post-administration, 3 hours post-administration, 5 hours post-administration, 10 hours post-administration, 12 hours post-administration, or 24 hours post-administration. In some cases, a target concentration of omega-3 fatty acid is maintained for greater than one hour post-administration, 3 hours post-administration, 5 hours post-administration, 10 hours post-administration, 12 hours post-administration, or 24 hours post-administration.

### Certain Definitions

As used herein and in the appended claims, the singular forms “a,” “and,” and “the” include plural referents unless the context clearly dictates otherwise. Thus, for example, reference to “an agent” includes a plurality of such agents, and reference to “the cell” includes reference to one or more cells (or to a plurality of cells) and equivalents thereof known to those skilled in the art, and so forth. When ranges are used herein for physical properties, such as molecular weight, or chemical properties, such as chemical formulae, all combinations and subcombinations of ranges and specific embodiments therein are intended to be included. The term “about” when referring to a number or a numerical range means that the number or numerical range referred to is an approximation within experimental variability (or within statistical experimental error), and thus the number or numerical range may vary between 1% and 15% of the stated number or numerical range. The term “comprising” (and related terms such as “comprise” or “comprises” or “having” or “including”) is not intended to exclude that in other certain embodiments, for example, an embodiment of any composition of matter, composition, method, or process, or the like, described herein, may “consist of” or “consist essentially of” the described features.

“Prevention” is the action of stopping something from happening or arising.

“Progression” is the process of developing or moving gradually towards a more advanced state

“Treating” or “treatment” as used herein includes any approach for obtaining beneficial or desired results in a subject's condition, including clinical results. Beneficial or desired clinical results can include, but are not limited to, alleviation or amelioration of one or more symptoms or conditions, diminishment of the extent of a disease, stabilizing (i.e., not worsening) the state of disease, delay or slowing of disease progression, amelioration, diminishment of the reoccurrence of disease. Treatment may prevent the disease from occurring; relieve the disease's symptoms, fully or partially remove the disease's underlying cause, shorten a disease's duration, or do a combination of the above.

“Treating” and “treatment” as used herein may also include prophylactic treatment. Treatment methods include administering to a subject a therapeutically effective amount of an active agent. The administering step may consist of a single administration or may include a series of administrations. The length of the treatment period depends on a variety of factors, such as the severity of the condition, the age of the patient, the concentration of active agent, the activity of the compositions used in the treatment, or a combination thereof. It will also be appreciated that the effective dosage of an agent used for the treatment or prophylaxis may increase or decrease over the course of a particular treatment or prophylaxis regime. Changes in dosage may result and become apparent by standard diagnostic assays known in the art. In some instances, chronic administration may be required. For example, the compositions are administered to the subject in an amount and for duration sufficient to treat the patient.

The terms “effective amount,” “therapeutically effective amount” or “pharmaceutically effective amount” refer to an amount of an active agent effective to treat a disease, such as brain or CNS diseases, systemic diseases or other diseases, including a range of effects, from a detectable amount of improvement to substantial relief/improvement of symptoms or a cure of the disease or condition. The result can be a reduction and/or alleviation of the signs, symptoms, or causes of a disease, or any other desired alteration of a biological system. For example, an “effective amount” for therapeutic uses is the amount of the composition comprising an agent as set forth herein required to provide a clinically significant decrease in an ophthalmic disease. For example, for the given aspect (e.g., length of incidence), a therapeutically effective amount will show an increase or decrease of at least 5%, 10%, 15%, 20%, 25%, 40%, 50%, 60%, 75%, 80%, 90%, or 100%. Therapeutic efficacy can also be expressed as “-fold” increase or decrease. For example, a therapeutically effective amount can have at least a 1.2-fold, 1.5-fold, 2-fold, 5-fold, or more effect over a control. An appropriate “effective” amount in any individual case may be determined using techniques, such as a dose escalation study.

The term “periorbital” refers to the area surrounding the socket of the eye.

The term “preorbital” refers to the area in front of the orbit or eye socket.

The term “eyelid” refers to movable folds of the skin over the eye.

“OD” refers to the right eye.

“OS” refers to the left eye.

“OU” refers to both eyes.

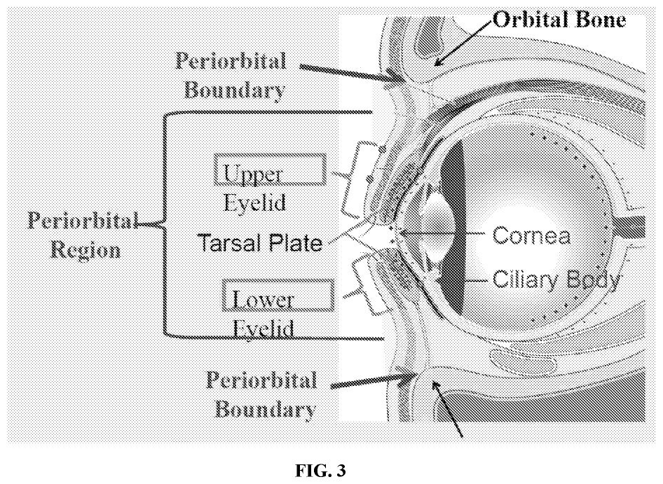

“Periorbital administration” involves administration to the periorbital skin (as depicted in FIG. 3) and specifically excludes administration to the upper eyelid, lower eyelid, and eyelid margins.

The term “lotion” describes an emulsion liquid dosage form. This dosage form is generally for external application to the skin (US FDA Drug Nomenclature Monograph, number C-DRG-00201).

The term “cream” describes an emulsion semisolid dosage form, usually containing >20% water and volatiles and/or <50% hydrocarbons, waxes or polyols as the vehicle. A cream is more viscous than a lotion. This dosage form is generally for external application to the skin (US FDA Drug Nomenclature Monograph, number C-DRG-00201).

The term “ointment” describes a semisolid dosage form, usually containing <20% water and volatiles and/or >50% hydrocarbons, waxes or polyols as the vehicle. This dosage form is generally for external application to the skin or mucous membranes (US FDA Drug Nomenclature Monograph, number C-DRG-00201).

The term “solution” describes a clear, homogeneous liquid dosage form that contains one or more chemical substances dissolved in a solvent or mixture of mutually miscible solvents (US FDA Drug Nomenclature Monograph, number C-DRG-00201).

The term “suspension” refers to a heterogeneous mixture containing solid particles that are sufficiently large for sedimentation.

“Emulsion” means, but is not limited to, an oil-in-water emulsion, a water-in-oil emulsion, a micro emulsion referring to particle sizes of 10-9.

“Formulation” and “composition,” are intended to be equivalent and refer to a composition of matter suitable for pharmaceutical use (i.e., producing a therapeutic effect as well as possessing acceptable pharmacokinetic and toxicological properties).

“Emollient” is an agent that softens and soothes the skin.

“Humectant” is a hygroscopic agent that moistens the skin.

“Penetration enhancer” is an agent that improves transdermal drug delivery.

“Thickening agent” increases the viscosity of a formulation to achieve optimal application characteristics.

“Pharmaceutically acceptable” is used as equivalent to physiologically acceptable. In certain embodiments, a pharmaceutically acceptable composition or preparation will include agents for buffering and preservation in storage, and can include buffers and carriers for appropriate delivery, depending on the route of administration.

The terms “subject,” “patient,” “individual,” are not intended to be limiting and can be generally interchanged. That is, an individual described as a “patient” does not necessarily have a given disease, but may be merely seeking medical advice. The term “subject” as used herein includes all members of the animal kingdom prone to suffering from the indicated disorder. In some aspects, the subject is a mammal, and in some aspects, the subject is a human.

The term “combination” refers to separate entities used together to achieve improved or optimal therapeutic benefit and safety. In the simplest case, the combination may be a combination of two therapeutic agents at fixed doses administered concomitantly. In this case, the ingredients may be separately formulated or mixed together in a single formulation. However, to achieve satisfactory disease control, the doses of the therapeutic agents and the relative timing of their administration may require a degree of flexibility. For example, in a combination of two therapies, one therapy of the two may be administered first to establish its baseline level of remediation before the other (second) drug is added. A combination of drugs may involve administration of drugs by different formulations, dosing methods, and different routes of administration.

The IP receptor is a cell surface protein that belongs to the G protein coupled receptor superfamily. The primary endogenous ligands for the IP receptor are prostacyclin (PGI2), prostaglandin E₁ (PGE1), and 19(S)-HETE (Woodward D, et al. (2011) Pharmacol Rev 63:471-538; Tunara S et al. (2016) PLOS one 11:0163633).

The platelet activating factor (PAF) receptor is also is a cell surface protein that belongs to the G protein coupled receptor superfamily (Ishii S et al. (2002) PGs & Other Lipid Med 68-69:599-609).

SiRNA represents the same entity variously described as small interfering RNA, small inhibitory RNA, and short interfering RNA.

The term “macromolecule” as used herein refers to a molecule with a size of about 100 to 10,000 angstroms, or with a molecular weight exceeding about 1,500 daltons. The term macromolecule comprises peptides, proteins, nucleic acids (RNA and DNA), oligonucleotides, carbohydrates, lipids/fats, oligonucleotides, and viral vectors used in gene therapy, for example.

### EXAMPLES

### Example 1: Cynomolgus Monkey Study of JV-DE1 Administered to the Periorbital Skin of One Eye

An experiment was performed to determine the biodisposition of 4,5-dihydro-N-[4-[[4-(1-methylethoxy) phenyl]methyl] phenyl]-1H-imadazol-2-amine (JV-DE1) following topical application to the periorbital skin that surrounds the anterior portion of the globe was performed. Results are summarized in Table 1.

TABLE 1
Biodistribution of JV-DE1 in a dosed eye (OD) vs. undosed
eye (OS) following delivery by NIODP in Cynomolgus monkeys

| Time Post Dose (hour) | | | 0 | 0.5 | 3 | 6 | 24 |
|---|---|---|---|---|---|---|---|
| JV-DE1 | Plasma (ng/mL) | | BLQ | 0.131 | 0.21 | 0.26 | BLQ |
| Tissue (ng/g) | OD | Upper eyelid | NA | 26535.4 | 32454.7 | 7945.6 | 11597.8 |
| Fluid (ng/mL) | | Cornea | NA | 9299.3 | 16688.7 | 23929.5 | 1621.3 |
| Dosed: | | Retina | NA | 39.6 | 27.9 | 45.5 | 5.5 |
| 165.4 μg (OD) | | Vitreous humor | NA | 15.2 | 7.79 | 3.9 | 18.97 |
| LLOQ = | | Retina/Upper | NA | 0.1% | 0.1% | 0.6% | 0.0% |
| 0.1 ng/ml | | eyelid | | | | | |
| | OS | Upper eyelid | NA | 733.4 | 1637 | 378.2 | 46.3 |
| | | Cornea | NA | 0 | 0 | 0 | 0 |
| | | Retina | NA | 5.2 | 6.3 | 4.8 | 1.4 |
| | | Vitreous humor | NA | 1.03 | 1.85 | 0.57 | 0 |
| | | Retina/Upper | NA | 0.7% | 0.4% | 1.3% | 3.0% |
| | | eyelid | | | | | |
| | OS/OD | Retina | NA | 13.1% | 22.6% | 10.5% | 25.5% |

165.4 μg of JV-DE1, a synthesized chemical compound, was applied to the right eye (OD) of Cynomolgus monkeys via NIODP. The monkeys were restrained from rubbing their eyes for 2 hours post dose and closely monitored by experimenter(s) to prevent cross-contamination of the untreated eye, yet JV-DE1 was quickly found in the undosed upper eyelid (733.4 ng/g), retina (5.2 ng/g) and Vitreous humor (1.03 ng/mL) as early as 0.5-hour post dose. Throughout the 3, 6, and 24-hour post dose points, low levels of JV-DE1 was detected in the retina at 5.5 ˜ 45.5 ng/g of the dosed eye, and 1.4 ˜ 6.3 ng/g of the undosed eye. The ratio of JV-DE1 in undoseD retina vs. the dosed retina was below 25.5% at all post dose time points tested. Compared to the dosed periorbital skin (JV-DE1 Cmax 32454.7 ng/g), lower but substantial JV-DE1 levels were detected in the periorbital skin of the undosed eye (Cmax 1637.0 ng/g). These observations suggest that the efflux transporters in the CNS limit the uptake of JV-DE1, and the forward TORT pathway (solid black arrows, FIG. 2) seems to provide a fast one-way-out route for JV-DE1 to be quickly expelled out of the CNS to various outlets nearby, such as the non-CNS tissues along the forward TORT pathway, including the ocular tissues/muscles of the undosed eye, the CSF, and/or blood. for terminal clearance.

### Example 2: Cynomolgus Monkey Study of Docosahexaenoic Acid Administered Periorbitally

An experiment was performed to determine the biodisposition of DHA following topical application to the periorbital skin that surrounds the anterior portion of the globe was performed. Results are summarized in Table 2.

TABLE 2
Biodistribution of DHA in dosed eye (OS) v. undosed eye
(OD) following delivery by NIODP in Cynomolgus monkeys

| Time Post Dose (hour) | | | 0 | 0.5 | 3 | 6 | 24 |
|---|---|---|---|---|---|---|---|
| DHA | Plasma (μg/mL) | | 2.4 | 2.2 | 3.1 | 2.1 | 1.2 |
| Monkey | OD | Upper eyelid | BLQ | 41.1 | 5.8 | 7.4 | 5.3 |
| Tissue (μg/g) | | Cornea | BLQ | 0 | 0 | 0 | 0 |
| Fluid (μg/mL) | | Retina | 16.5 | 70.8 | 97.3 | 78.2 | 35.6 |
| Dosed (OS): | | Vitreous humor | BLQ | 0 | 0 | 0 | 0 |
| 6755.1 μg | | Retina/Upper | NA | 172% | 1678% | 1057% | 672% |
| LLOQ = | | eyelid | | | | | |
| 0.5 μg/mL | OS | Upper eyelid | BLQ | 380.6 | 419.6 | 10.7 | 16.2 |
| | | Cornea | BLQ | 66.3 | 21.6 | 22.3 | 25.9 |
| | | Retina | 16.5 | 110.9 | 66.7 | 78.6 | 47.3 |
| | | Vitreous humor | BLQ | 0 | 0 | 0 | 0 |
| | | Retina/Upper | NA | 29% | 16% | 735% | 292% |
| | | eyelid | | | | | |
| | OD/OS | Retina | 100% | 64% | 146% | 99% | 75% |

Docosahexaenoic acid (DHA) is an omega-3 fatty acid and an essential component for neuronal formation, function and protection. 6775.1±92.5 (mean±SEM) μg/eye of DHA was applied to the left eye (OS) of Cynomolgus monkeys via NIODP. DHA was detected in the upper eyelid (41.1 μg/g) and retina (70.8 μg/g) of the undosed OD as soon as 0.5-hour post dose, which was unlikely caused by cross contamination due to eye rubbing, because the monkeys were restrained from rubbing their eyes for 2 hours post dose and closely monitored by experimenter(s). Throughout the 3, 6, and 24-hour post dose points, DHA was detected in the undosed eye, at high concentrations in the upper eyelid (from 5.3 to 41.1 μg/g) but slower than in the dosed eye. High concentrations of DHA (from 35.6 to 97.3 μg/g) were detected in the undoes retina, which is similar to the dosed retina. No DHA was detectable in the undosed right cornea and vitreous humor.

The distribution pattern of DHA in the undosed eye is different from that of the dosed eye in three aspects: (1) significantly higher retina/upper eyelid ratio of DHA concentration: 172% to 1678% in the undosed eye vs. 29% to 735% in the dosed eye; (2) a constant concentration equilibrium observed between the dosed and undosed retina, with the drug levels sometimes higher in the undosed retina: 64% to 146% of the dosed one; (3) reversed concentration gradient of retina vs. upper eyelid and obvious absence of cornea pathway in the undosed eye: the biodistribution gradient of the undosed OD was retina>>eyelid>>cornea=vitreous humor=0. Moreover, compared to the baseline retina/plasma ratio of 7-fold, and with no significant changes in plasma, the retina/plasma ratios of DHA in the undosed eye were at least 30-fold at each post dose timepoint, signifying that the undosed retina uptake of DHA was not from blood circulation.

The fast, high dose, and constantly equilibrated trans-retinal biodistribution, and significantly higher retina/upper eyelid ratio of DHA concentration in the undosed eyeare contrast to the pattern of JV-DE1 between the undosed and undosed retina. DHA may use the TORT and rev-TORT pathway for recycling between the left and right retina tissues through the optic nerve, and within the brain to exert its physiological functions. More brain concentration should be achieved by using high purity (>90%) DHA to delivery about 30 mg/eye DHA to both eyes. In a normal healthy person, the gray matter of the brain and photoreceptor cells of the retina contain 30-40% of DHA as phospholipids. In addition to being a major structural element of neuronal membranes, high concentration of DHA is also found in synaptic membranes, mitochondria and microsomes at the subcellular level. DHA regulates fluidity, permeability and viscosity of synaptic membranes, modulates neurotransmission and gene expression, and activates enzymes, receptors and ion channels. The existence of fatty acid transporter proteins (FATPs), fatty acid binding proteins (FABPs), and receptors for DHA, as well as its release at synapses suggests that DHA is not only an essential nutrient for synaptogenesis and neurotransmission, but also an important cargo in synaptic transmission, and is involved in neuronal signaling in the brain.

### Example 3: Endogenous DHA Levels in Ocular Tissues

Table 3 shows the endogenous levels of DHA in monkey ocular tissues were mostly below the lower limit of quantification (LLOQ=0.5 μg/mL) or negligible. The endogenous levels DHA is originated from normal diet and not attributable to NIODP administration. This is to confirm the significantly increased biodistribution of DHA in monkey ocular tissues (including tissues in the undosed eye) following periorbital skin administration, shown in Table 2, is not due to endogenous levels of DHA.

TABLE 3
Baseline Endogenous Levels of DHA in Monkeys

| | | Calculated Concentration |
|---|---|---|
| Sample Name | Sample Type | (μg/g, μg/mL for fluid) |
| Double blank-1-retina | Double Blank | 16 |
| Double blank-2-retina | Double Blank | 17 |
| Double blank-1-upper | Double Blank | BLQ |
| eyelid | | |
| Double blank-2-upper | Double Blank | BLQ |
| eyelid | | |
| Double blank-1-cornea | Double Blank | BLQ |
| Double blank-2-cornea | Double Blank | BLQ |
| Double blank-1-plasma | Double Blank | 0.5 |
| Double blank-2-plasma | Double Blank | 0.6 |
| Double blank-1-vitreous | Double Blank | BLQ |
| humor | | |
| Double blank-2-vitreous | Double Blank | BLQ |
| humor | | |

BLQ: Below the lower limit of quantification (LLOQ = 0.5 μg/mL) and given a value of 0 in relevant calculations.

### Example 4: Baseline Endogenous Levels of DHA, DHA-EE, and JV-DE1 in Bama Minipigs

Table 4 shows the endogenous levels of DHA, DHA, EE, and JV-DE1 in Bama minipigs. Endogenous levels were measured in the plasma, cerebrospinal fluid, ocular tissues (retina, optic nerve, ocular muscles), and brain. The endogenous levels DHA are not attributable to NIODP administration of DHA, DHA-EE, or JV-DE1. The baseline data collected in Table 4 was taken from the same lot of pigs, from the same vendor, as the information collected in Tables 5-7.

TABLE 4
Baseline Endogenous Levels of DHA, DHA-EE, and JV-DE1 in Bama Minipigs

| | DHA | | DHA-EE | | JV-DE1 | |
|---|---|---|---|---|---|---|
| | Concentration | | Concentration | | Concentration | |
| | Tissue (ng/g) | LLOQ | Tissue (ng/g) | LLOQ | Tissue (ng/g) | LLOQ |
| Analyte | Fluid (ng/mL) | (ng/mL) | Fluid (ng/mL) | (ng/mL) | Fluid (ng/mL) | (ng/mL) |
| Plasma | 122.1 | 100 | BLQ | 5 | BLQ | 0.1 |
| Cerebrospinal fluid | BLQ | 50 | BLQ | 100 | BLQ | 0.025 |
| Eye tissue homogenate | 2643.5 | 100 | BLQ | 15 | BLQ | 0.025 |
| including (Retina, optic | | | | | | |
| nerve, ocular muscle) | | | | | | |
| Brain | 20758 | 100 | BLQ | 15 | BLQ | 0.025 |

### Example 5: Docasahexaenoic Acid (DHA) in Bama Minipigs Following Single Periorbital Skin Administration

In this example, 6092 μg/eye of DHA was applied to the periorbital skin of a left eye of a Bama minipig. As shown in Table 5, at three hours post-dosage, DHA was detected in both the left and right halves of the brain of the pig. 27133.8 ng/g of tissue and 24795.8 ng/g of tissue of DHA was measured in the left and right halves of the brain, respectively. Taking the average of these two numbers, the amount of DHA present in the brain 3 hours post-dosage was 25964.9 ng/g. As shown in Table 4, endogenous levels of DHA in this species of pig were measured to be 20758 ng/g of brain. Therefore, this experiment showed that DHA levels in the brain were increased by 5206.8 ng/g or 25% three hours after periorbital administration of DHA.

Given that the average Bama pig brain weighs around 60 g, the amount of DHA delivered to the brain can be estimated to be 312,408 ng or 312 μg. Therefore, the percentage of administered dose=312 μg delivered to brain/6092 μg dosed=5.1% of DHA topically administered to the pig via periorbital administration was delivered to the brain. As a comparison, only about 0.025% of DHA that is orally administered is delivered to the brain or CNS. These results show that periorbital or eyelid skin administration of topical DHA formulations provides an efficient platform for delivering DHA to the brain.

Additionally, as shown in Table 5, an increased concentration of DHA was also measured in the ocular muscles, of the dosed more than the undosed eye. This may be beneficial for DHA delivery via NIODP to intermediate ocular segments to treat related diseases, such as intermediate uveitis.

TABLE 5
Result of Docosahexaenoic acid in Bama minipigs
Following Single Periorbital Skin Administration

| | | Test Article | | |
|---|---|---|---|---|
| | | Docosahexaenoic | | |
| | | acid (DHA) | | |
| | | Dosing site | | |
| | | Periorbital skin | | |
| | | Animal No. | | |
| | | 201 | | |
| | | Time (h) | | |
| | | 0 | 1 | 3 |
| | | Dose Level (μg/left eye) | | |
| | | 6092 | | |
| | | Dose Level (μg/right eye) | | |
| | | NA | | |
| | | Drug Concentration | | |
| | | in Plasma (ng/ml) | | |
| | | BLQ | BLQ | BLQ |
| Drug | brain-left | | | 27133.8 |
| Concentration | brain-right | | | 24795.8 |
| in Tissue | retina-left | | | 61713.7 |
| (ng/g) | retina-right | | | 42141.2 |
| | optic nerve-left | | | 10437.1 |
| | optic nerve-right | | | 15253.5 |
| | inferior rectus muscle-left | | | 2754.6 |
| | inferior rectus muscle- | | | 2342.6 |
| | right | | | |
| | medial rectus muscle-left | | | 2717.3 |
| | medial rectus muscle-right | | | 1523.9 |
| | lateral rectus muscle-left | | | 8096.7 |
| | lateral rectus muscle-right | | | 1979.4 |
| | superior rectus muscle-left | | | 3052.7 |
| | superior rectus muscle- | | | 0 |
| | right | | | |
| | Cerebrospinal fluid* | | | 0 |
| % of | brain-left | | | 6% |
| administered | brain-right | | | 4% |
| dose | retina-left | | | 0.2% |
| | retina-right | | | 0.1% |
| | optic nerve-left | | | 0.03% |
| | optic nerve-right | | | 0.05% |
| | inferior rectus muscle-left | | | 0.004% |
| | inferior rectus muscle- | | | 0.01% |
| | right | | | |
| | medial rectus muscle-left | | | 0.01% |
| | medial rectus muscle-right | | | 0.01% |
| | lateral rectus muscle-left | | | 0.02% |
| | lateral rectus muscle-right | | | 0.03% |
| | superior rectus muscle-left | | | 0.01% |
| | superior rectus muscle- | | | 0% |
| | right | | | |
| | Cerebrospinal fluid | | | 0% |

BLQ: Below the limit of quantification which was calculated as 0; LLOQ = 100 ng/ml

### Example 6: Docasahexaenoic Acid Ethyl Ester (DHA-EE) in Bama Minipigs Following Single Periorbital Skin Administration

In this example, 6174 μg/eye of docohexaenoic acid ethyl ester (DHA-EE) was applied to the periorbital skin of a left eye of a Bama minipig. As shown in Table 6, at three hours post-dosage, DHA was detected in both the left and right halves of the brain of the pig. 21099.7 ng/g of tissue and 21336.3 ng/g of tissue of DHA was measured in the left and right halves of the brain, respectively. At this same time point, no DHA-EE was measured in the brain but detectable in ocular muscles of the dosed eye, showing that after administration, DHA-EE is rapidly converted to DHA in the brain. The average amount of DHA present in the brain 3 hours post-dosage was 21218 ng/g. As shown in Table 4, endogenous levels of DHA in this species of pig were measured to be 20758 ng/g of tissue. Therefore, this experiment showed that DHA levels in the brain were increased by 460 ng/g or 2.2% from the baseline three hours after periorbital administration of DHA-EE.

Given that the average Bama pig brain weighs around 60 g, the amount of DHA delivered to the brain can be estimated to be 27,600 ng or 27.6 μg. Therefore, the percentage of administered dose=27.6 μg delivered to brain/6174 μg dosed=0.45% of DHA topically administered to the pig via periorbital administration was delivered to the brain. As a comparison, only about 0.025% of DHA that is orally administered is delivered to the brain or CNS. These results show that periorbital or eyelid skin administration of DHA-EE also provides an efficient platform for delivering DHA to the brain.

TABLE 6
Result of Docosahexaenoic acid Ethyl Ester in Bama minipigs
Following Single Periorbital Skin Administration

| Test Article | | Docosahexaenoic acid ethyl ester (DHA-EE) | | | | | |
|---|---|---|---|---|---|---|---|
| Dosing site | | Periorbital skin | | | | | |
| Animal No. | | 101 | | | | | |
| Time (h) | | 0 | 1 | 3 | 0 | 1 | 3 |
| Dose Level (μg/left eye) | | 6174 | | | NA | | |
| Dose Level (μg/right eye) | | NA | | | NA | | |
| Analyte | | DHA-EE | | | DHA | | |
| Drug Concentration | | BLQ | BLQ | BLQ | BLQ | BLQ | BLQ |
| in Plasma (ng/mL) | | | | | | | |
| Drug | brain-left | | | 0 | | | 21099.7 |
| Concentration | brain-right | | | 0 | | | 21336.3 |
| in Tissue | retina-left | | | 0 | | | 48882.9 |
| (ng/g) | retina-right | | | 0 | | | 41629.4 |
| | optic nerve-left | | | 0 | | | 8794.6 |
| | optic nerve-right | | | | | | 8839.4 |
| | inferior rectus | | | 7444.8 | | | 3161.1 |
| | muscle-left | | | | | | |
| | inferior rectus | | | 0 | | | 1287.8 |
| | muscle-right | | | | | | |
| | medial rectus | | | 7382.3 | | | 4874.6 |
| | muscle-left | | | | | | |
| | medial rectus | | | 0 | | | 1690.7 |
| | muscle-right | | | | | | |
| | lateral rectus | | | 995.7 | | | 1356.3 |
| | muscle-left | | | | | | |
| | lateral rectus | | | 0 | | | 1085.6 |
| | muscle-right | | | | | | |
| | superior rectus | | | 12844.5 | | | 10400.7 |
| | muscle-left | | | | | | |
| | superior rectus | | | 0 | | | 743.6 |
| | muscle-right | | | | | | |
| | Cerebrospinal | | | 0 | | | 0 |
| | fluid*(mL) | | | | | | |
| % of | brain-left | | | 0% | | | 0.332% |
| administered | brain-right | | | 0% | | | 0.562% |
| dose | retina-left | | | 0% | | | 0.130% |
| | retina-right | | | 0% | | | 0.110% |
| | optic nerve-left | | | 0% | | | 0.011% |
| | optic nerve-right | | | 0% | | | 0.031% |
| | inferior rectus | | | 0.02% | | | 0.008% |
| | muscle-left | | | | | | |
| | inferior rectus | | | 0% | | | 0.009% |
| | muscle-right | | | | | | |
| | medial rectus | | | 0.02% | | | 0.020% |
| | muscle-left | | | | | | |
| | medial rectus | | | 0% | | | 0.016% |
| | muscle-right | | | | | | |
| | lateral rectus | | | 0.003% | | | 0.004% |
| | muscle-left | | | | | | |
| | lateral rectus | | | 0% | | | 0.010% |
| | muscle-right | | | | | | |
| | superior rectus | | | 0.04% | | | 0.043% |
| | muscle-left | | | | | | |
| | superior rectus | | | 0% | | | 0.005% |
| | muscle-right | | | | | | |
| | Cerebrospinal | | | 0% | | | 0% |
| | fluid*(mL) | | | | | | |

DHA LLOQ = 100 ng/mL;
DHA-EE LLOQ = 15 ng/mL
% of administered dose = (Drug Content in Tissue (ng))/Dose Level (ng/eye) *100

### Example 7:4,5-dihydro-N-[4-[[4-(1-methylethoxy) phenyl]methyl] phenyl]-1H-imadazol-2-amine (JV-DE1) Delivery in Bama Minipigs Following Single Periorbital Skin Administration

In this example, 158.45 μg of 4,5-dihydro-N-[4-[[4-(1-methylethoxy) phenyl]methyl] phenyl]-1H-imadazol-2-amine (JV-DE1) was applied to the periorbital skin of a right eye of a Bama minipig. As shown in Table 7, at three hours post-dosage, minimal amounts of JV-DE1 were detected in both the left and right halves of the brain of the pig. 1.37 ng/g of tissue and 1.35 ng/g of tissue of JV-DE1 was measured in the left and right halves of the brain, respectively. The average amount of JV-DE1 present in the brain 3 hours post-dosage was 1.36 ng/g. As shown in Table 4, endogenous levels of JV-DE1 in this species of pig were below the lower limit of quantification.

Given that the average Bama pig brain weighs around 60 g, the amount of JV-DE1 delivered to the brain can be estimated to be 80.6 ng. Therefore, 0.051% of the 158.45 μg of JV-DE1 administered to the periorbital skin was delivered to the brain, much less than the deliverability of DHA via NIODP. As a comparison, only about 0.025% of DHA that is orally administered is delivered to the brain or CNS. These results show that periorbital or eyelid skin administration may generally achieve better percentage of administered dose than oral administration for brain delivery, even for “unfavored” compounds like JV-DE1.

TABLE 7
Result of JV-DE1 in Bama minipigs Following
Single Periorbital Skin Administration

| | | Test Article | | |
|---|---|---|---|---|
| | | JV-DE1 | | |
| | | Dosing site | | |
| | | Periorbital skin | | |
| | | Animal No. | | |
| | | 101 | | |
| | | Time (h) | | |
| | | 0 | 1 | 3 |
| | | Dose Level (μg/left eye) | | |
| | | NA | | |
| | | Dose Level (μg/right eye) | | |
| | | 158.45 | | |
| | | Drug Concentration | | |
| | | in Plasma (ng/ml) | | |
| | | BLQ | BLQ | BLQ |
| Drug | brain-left | | | 1.37 |
| Concentration | brain-right | | | 1.35 |
| in Tissue | retina-left | | | 2.30 |
| (ng/g) | retina-right | | | 2.70 |
| | optic nerve-left | | | 11.94 |
| | optic nerve-right | | | 33.63 |
| | inferior rectus muscle-left | | | 57.27 |
| | inferior rectus muscle-right | | | 58.71 |
| | medial rectus muscle-left | | | 43.67 |
| | medial rectus muscle-right | | | 65.54 |
| | lateral rectus muscle-left | | | 53.65 |
| | lateral rectus muscle-right | | | 43.50 |
| | superior rectus muscle-left | | | 85.83 |
| | superior rectus muscle-right | | | 88.37 |
| | Cerebrospinal fluid*(μL) | | | BLQ |
| % of | brain-left | | | 0.0518% |
| administered | brain-right | | | 0.0513% |
| dose | retina-left | | | 0.0002% |
| | retina-right | | | 0.0003% |
| | optic nerve-left | | | 0.0006% |
| | optic nerve-right | | | 0.0047% |
| | inferior rectus muscle-left | | | 0.0061% |
| | inferior rectus muscle-right | | | 0.0170% |
| | medial rectus muscle-left | | | 0.0071% |
| | medial rectus muscle-right | | | 0.0260% |
| | lateral rectus muscle-left | | | 0.0063% |
| | lateral rectus muscle-right | | | 0.0175% |
| | superior rectus muscle-left | | | 0.0140% |
| | superior rectus muscle-right | | | 0.0237% |
| | Cerebrospinal fluid*(μL) | | | 0% |

*This tissue was taken directly for analysis without other homogenate solvents.
Homogenate dilution factor = Homogenization Solvent volume/tissue weight + 1.
BLQ: Below the limit of quantification which was calculated as 0; LLOQ = 0.025 ng/ml
Drug Content in Tissue (ng) = Drug Concentration in Tissue (ng/g) * (Tissue weight (mg)/1000)
% of administered dose = (Drug Content in Tissue (ng))/Dose Level (ng/eye) * 100
Tissue/plasma ratio (mL/g) = Tissue drug concentration (ng/g)/plasma drug concentration (ng/mL).
NC: Not calculable (the tissue was not the administered site or the drug concentration in plasma was tested as BLQ).

### Example 8: JV-DE1 Distribution in New England Rabbits Following Single Periorbital Skin Administration

NIODP may be more efficient than topical eye drops for corneal delivery to treat corneal disorders, such as dry disease. As shown in Table 1, the Cmax of JV-DE1 is 23929.5 ng/g in monkey cornea by delivery of 165.4 μg per eye via NIODP, while the Cmax is only 12124.85 ng/g in rabbit cornea when 120 μg/eye of JV-DE1 was delivered as eye drops (as shown in Table 8). Accordingly, NIODP may be used to deliver other compounds (such as DHA) to the cornea to treat corneal diseases, such as dry eye.

TABLE 8
Result of JV-DE1 in New Zealand Rabbits
Following Single Eye Drop Administration

| | | | Animal | Time | Cornea | Retina |
|---|---|---|---|---|---|---|
| Compound | Group | Site | ID | (h) | ng/g | |
| JV-DE1 | Rabbit | OD | 2036281 | 0.5 | 12124.85 | 92.03 |
| | 120 μg/eye | | 2036282 | 2 | 5952.6 | 49.13 |
| | Eye drops | | 2036283 | 6 | 5125.52 | 58.87 |
| | to the | | 2036284 | 24 | 542.19 | 6.82 |
| | right eye | OS | 2036281 | 0.5 | 78.35 | 7.35 |
| | (LLOQ 0.1 | | 2036282 | 2 | 28.92 | 3.38 |
| | ng/mL) | | 2036283 | 6 | 67.82 | 1.42 |
| | | | 2036284 | 24 | 11.03 | BLQ |

While preferred embodiments of the present invention have been shown and described herein, it will be obvious to those skilled in the art that such embodiments are provided by way of example only. It is not intended that the invention be limited by the specific examples provided within the specification. While the invention has been described with reference to the aforementioned specification, the descriptions and illustrations of the embodiments herein are not meant to be construed in a limiting sense. Numerous variations, changes, and substitutions will now occur to those skilled in the art without departing from the invention. Furthermore, it shall be understood that all aspects of the invention are not limited to the specific depictions, configurations or relative proportions set forth herein which depend upon a variety of conditions and variables. It should be understood that various alternatives to the embodiments of the invention described herein may be employed in practicing the invention. It is therefore contemplated that the invention shall also cover any such alternatives, modifications, variations or equivalents. It is intended that the following claims define the scope of the invention and that methods and structures within the scope of these claims and their equivalents be covered thereby.

1. A method of promoting health, preventing or treating disease in the central nervous system (CNS) of a subject, comprising administering a topical pharmaceutical composition comprising an active agent to periorbital skin or eyelid of an eye of said subject.

2. The method of claim 1, wherein said active agent is omega-3 fatty acid (Omega-3 FA), Omega-3 triglycerides (omega-3 TG), and Omega-3 ethyl esters (Omega-3 EE), or a pharmaceutically acceptable ester or salt thereof.

3. The method of claim 2, wherein said omega-3 fatty acid (Omega-3 FA), Omega-3 triglycerides (omega-3 TG), or Omega-3 ethyl esters (Omega-3 EE), or a pharmaceutically acceptable ester or salt thereof, is synthesized, is isolated from a plant source, or is isolated from fish tissue.

4. The method of claim 2, wherein said omega-3 fatty acid comprises alpha-linolenic acid (ALA), eicosapentaenoic acid (EPA), docosahexaenoic acid (DHA), or any combination thereof.

5. (canceled)

6. The method of claim 4, wherein said topical pharmaceutical composition comprises greater than 50% DHA.

7.-10. (canceled)

11. The method of claim 1, wherein said topical pharmaceutical composition comprises at least one of: petrolatum; shea butter, lanolin, or Vitamin E.

12.-15. (canceled)

16. The method of claim 2, wherein said omega-3 fatty acid is administered in an amount of from about 0.1 mg to about 3000 mg.

17. The method of claim 2, wherein said omega-3 fatty acid is administered in an amount of 5 to 200 mg per eye.

18. The method of claim 1, wherein said topical pharmaceutical composition is formulated as a cream, emulsion, foam, gel, ointment, or oil solution, and optionally includes an emollient, a humectant, a thickening agent, a preservative, a penetration enhancer, an anti-oxidant, an odor masking agent, or any combination thereof.

19. (canceled)

20. The method of claim 1, wherein said topical pharmaceutical composition further comprises a preservative.

21. The method of claim 1, wherein said topical pharmaceutical composition is free of preservatives.

22. The method of claim 1, wherein said topical pharmaceutical composition is administered with a bottle with a roller ball, a click pen brush, a pump bottle, or an eye drop bottle and Q-tip, an eye pad, or a fingertip.

23. The method of claim 1, wherein promoting health, preventing or treating disease in the CNS of a subject comprises treating or preventing addiction, amyotrophic lateral sclerosis, arachnoid cysts, attention deficit-hyperactivity disorder, autism, CNS injury, CNS tumors, catalepsy, encephalitis, epilepsy, meningitis, migraine, multiple sclerosis, myelopathy, Tourette's syndrome, Alzheimer's disease, Huntington's disease, or Parkinson's disease.

24. The method of claim 1, wherein said topical pharmaceutical composition is administered to said subject ranging from at least once daily, to once every seven days, and up to four times per day.

25.-27. (canceled)

28. The method of claim 2, wherein greater than 1:1 brain to plasma ratio of said omega-3 fatty acid administered to said periorbital skin or eyelid of said subject is delivered to a brain of said subject, where a greater than 1 brain to plasma ratio is generally considered high efficiency brain delivery.

29.-62. (canceled)

63. A pharmaceutical composition formulated for topical periorbital or eyelid administration, comprising an omega-3 fatty acid and one or more emollients.

64. The pharmaceutical composition of claim 63, wherein said omega-3 fatty acid is synthetic, isolated from fish tissue, or is isolated from a plant source.

65. (canceled)

66. The pharmaceutical composition of claim 63, wherein said omega-3 fatty acid comprises alpha-linolenic acid (ALA), eicosapentaenoic acid (EPA), docosahexaenoic acid (DHA), or any combination thereof.

67.-72. (canceled)

73. The pharmaceutical composition of claim 63, further comprising one or more emollients selected from petrolatum, shea butter, lanolin, or Vitamin E.

74.-77. (canceled)

78. The pharmaceutical composition of claim 63, wherein said pharmaceutical composition is formulated as a cream, emulsion, foam, gel, ointment, or oil solution, and optionally includes a humectant, a thickening agent, a preservative, a penetration enhancer, an anti-oxidant, an odor masking agent, or any combination thereof.

79. (canceled)

80. The pharmaceutical composition of claim 63, wherein said pharmaceutical composition further comprises a preservative.

81. The pharmaceutical composition of claim 63, wherein said pharmaceutical composition is free of preservatives.

82.-91. (canceled)

92. The pharmaceutical composition of claim 63, wherein said pharmaceutical composition further comprises greater than 3000 mg of said omega-3 fatty acid.

93.-125. (canceled)
